(19) United States
(12) Patent Application Publication
(10) Pub. No.: US 2011/0200573 A1
(43) Pub. Date: Aug. 18, 2011

(54) Methods of inhibiting retrovirus replication and infectivity

(76) Inventors: Derya Unutmaz, New York, NY (US); Vyacheslav Furtak, Nashville, TN (US)

(21) Appl. No.: 12/931,608

(22) Filed: Feb. 4, 2011

Related U.S. Application Data
(60) Provisional application No. 61/301,594, filed on Feb. 4, 2010.

(51) Int. Cl. A61K 38/43; C12N 9/00; A61P 31/18; C40B 30/00

(52) U.S. Cl. 424/94.1; 435/183; 506/7

(57) ABSTRACT

The present invention provides methods of inhibiting a retrovirus replication or infectivity by administering a therapeutically effective amount of MOV10 or a fragment, derivative or analog thereof or an inhibitor of MOV10 or a fragment, derivative or analog thereof or of inhibiting a retrovirus replication or infectivity by increasing or decreasing transcription, translation or biological activity of MOV10 or a fragment, derivative or analog thereof. In some instances the retrovirus is HIV or HIV-1. The present invention also features methods of treating a disease caused all or in part by a retrovirus, such as HIV or HIV-1, by administering a therapeutically effective amount of MOV10 or a fragment, derivative or analog thereof or an inhibitor thereof or by increasing or decreasing transcription, translation or biological activity of MOV10 or a fragment, derivative or analog thereof. Further, the present invention features methods to identify an agent that may inhibit a retrovirus comprising identifying an agent that is regulated by MOV10 or a fragment, derivative or analog thereof.

### CROSS REFERENCE TO RELATED APPLICATIONS

The present application claims the benefit under 35 U.S.C. §119(e) of U.S. provisional application Ser. No. 61/301,594, filed Feb. 4, 2010, the disclosure of which is herein incorporated by reference.

### GOVERNMENT SUPPORT

The research leading to the present inventions was funded in part by Grant No. R21AI078839-02 from the National Institutes of Health. The government may have certain rights in the invention.

### FIELD OF THE INVENTION

The present invention relates generally to inhibiting replication of retroviruses and inhibiting infection of target cells by retroviruses such as, for example, HIV-1, HIV-2 and SIV. Further, the present invention relates to agents that regulate replication of and infectivity of retroviruses such as HIV-1, HIV-2 and SIV. Furthermore, the present invention relates to methods of identifying agents that can modulate the infectivity of retroviruses such as HIV-1, HIV-2 and SIV.

### BACKGROUND OF THE INVENTION

A retrovirus is an RNA virus that is replicated in a host cell via the enzyme reverse transcriptase to produce DNA from its RNA genome. The DNA is then incorporated into the host's genome by an integrase enzyme. The virus thereafter replicates as part of the host cell's DNA. Retroviruses are enveloped viruses that belong to the viral family Retroviridae. A special variant of retroviri are endogenous retroviri which are integrated into the genome of the host and inherited across generations. The virus itself stores its nucleic acid in the form of a +mRNA (including the 5′cap and 3′PolyA inside the virion) genome and serves as a means of delivery of that genome into cells it targets as an obligate parasite, and constitutes the infection. Once in the host's cell, the RNA strands undergo reverse transcription in the cytoplasm and are integrated into the host's genome, at which point the retroviral DNA is referred to as a provirus. It is difficult to detect the virus until it has infected the host.

Simply, DNA is usually transcribed into RNA, and RNA is translated into protein. However, retroviruses function differently—their RNA is reverse-transcribed into DNA, which is integrated into the host cell's genome (when it becomes a provirus), and then undergoes the usual transcription and translational processes to express the genes carried by the virus. Therefore, the order of steps from a retroviral gene to a retroviral protein is: RNA→DNA→RNA→Protein.

Virions of retroviruses consist of enveloped particles about 100 nm in diameter. The virions also contain two identical single-stranded RNA molecules 7-10 kilobases (kb) in length. Although virions of different retroviruses do not have the same morphology or biology, all the virion components are very similar. The main virion components are the envelope composed of a protein capsid, which is obtained from the host plasma membrane during budding process. RNA consists of a dimer RNA. It has a cap at 5′ end and polyadenyle at 3′ end. The RNA genome also has terminal noncoding regions, which are important in replication, and internal regions that encode virion proteins for gene expression. The 5′ end includes four regions, which are R, U5, PBS, and L. R region is a short repeated sequence at each end of the genome during the reverse transcription in order to ensure correct end-to-end transfer in growing chain. U5, on the other hand, is a short unique sequence between R and PBS. PBS (primer binding site) consists of 18 bases complementary to 3′ end of tRNA primer. L region is an untranslated leader region that gives signal for packaging of genome RNA. The 3′ end includes 3 regions, which are PPT (polypurine tract), U3, and R. PPT is primer for plus-strand DNA synthesis during reverse transcription. U3 is a sequence between PPT and R, which has signal that provirus can use in transcription. R is the terminal repeated sequence at 3′ end. Proteins consist of gag proteins, protease (PR), pol proteins and env proteins. Gag proteins are major components of the viral capsid, which are about 2000-4000 copies per virion. Protease is expressed differently in different viruses. It functions in proteolytic cleavages during virion maturation to make mature gag and pol proteins. Pol proteins are responsible for synthesis of viral DNA and integration into host DNA after infection. Finally, env proteins play role in association and entry of virion into the host cell.

When retroviruses have integrated their own genome into the germ line, their genome is passed on to a following generation. These endogenous retroviruses (ERVs), contrasted with exogenous ones, now make up 5-8% of the human genome. Most insertions have no known function and are often referred to as “junk DNA.” However, many endogenous retroviruses play important roles in host biology, such as control of gene transcription, cell fusion during placental development in the course of the germination of an embryo, and resistance to exogenous retroviral infection. Endogenous retroviruses have also received special attention in the research of immunology-related pathologies, such as autoimmune diseases like multiple sclerosis, although endogenous retroviruses have not yet been proven to play any causal role in this class of disease.

While transcription was classically thought to only occur from DNA to RNA, reverse transcriptase transcribes RNA into DNA. The term “retro” in retrovirus refers to this reversal (making DNA from RNA) of the central dogma of molecular biology. Reverse transcriptase activity outside of retroviruses has been found in almost all eukaryotes, enabling the generation and insertion of new copies of retrotransposons into the host genome. These inserts are transcribed by enzymes of the host into new RNA molecules that enter the cytosol. Next, some of these RNA molecules are translated into viral proteins. For example, the gag gene is translated into molecules of the capsid protein, the pol gene is transcribed into molecules of reverse transcriptase, and the env gene is translated into molecules of the envelope protein. It is important to note that a retrovirus must “bring” its own reverse transcriptase in its capsid, otherwise it is unable to utilize the enzymes of the infected cell to carry out the task, due to the unusual nature of producing DNA from RNA.

Industrial drugs that are designed as protease and reverse transcriptase inhibitors can quickly be proved ineffective because the gene sequences that code for the protease and the reverse transcriptase can undergo many substitutions. These substitutions of nitrogenous bases, which make up the DNA strand, can make either the protease or the reverse transcriptase difficult to attack. The amino acid substitution enables the enzymes to evade the drug regiments because mutations in the gene sequences can cause physical or chemical change, which makes them harder to detect by the drug. When the drugs that are supposed to attack enzymes, such as protease, are designed, the manufacturers target specific sites on the enzyme. One way to attack these targets can be through hydrolysis of molecular bonds, which means that the drug will add molecules of H₂O (water) to specific bonds. By adding molecules of water at a site on the virus, the drug breaks the previous bonds that were linked to each other. If several of these breaks occur, the result can lead to lysis, the death of the virus.

Because reverse transcription lacks the usual proofreading of DNA replication, a retrovirus mutates very often. This enables the virus to grow resistant to antiviral pharmaceuticals quickly, and impedes the development of effective vaccines and inhibitors for the retrovirus.

One drawback of retroviruses, such as the Moloney retrovirus, involves the requirement for cells to be actively dividing for transduction. As a result, cells such as neurons are very resistant to infection and transduction by retroviruses. There is concern that insertional mutagenesis due to integration into the host genome might lead to cancer or leukemia. This is unlike Lentiviridae, a subclass of Retroviridae which are able to integrate their RNA into the genome of non-dividing host cells.

The human immunodeficiency viruses infect CD4⁺ macrophages and T helper cells. Although HIV-1 entry requires cell surface expression of CD4, to which the viral envelope glycoproteins bind, several studies have suggested that it is not sufficient for fusion of the viral envelope to the cellular plasma membrane. Early studies have shown that while human cells expressing a transfected CD4 gene were permissive for virus entry, murine cells expressing human CD4 were not. These findings led to the suggestion that there is a species-specific cell surface cofactor required in addition to CD4 for HIV-1 entry. Subsequent studies have shown that strains of HIV-1 that had been adapted for growth in transformed T-cell lines (T-tropic strains) could not infect primary monocytes or macrophages; in contrast, primary viral strains were found to infect monocytes and macrophages, but not transformed T cell lines. This difference in tropism was found to be a consequence of specific sequence differences in the gp120 subunit of the envelope glycoprotein, suggesting that multiple cell type-specific cofactors may be required for entry in addition to CD4.

The nature of the cofactors required for HIV entry proved elusive until it was recently discovered that the principal receptor for entry of macrophage-tropic (M-tropic) HIV-1 strains was CCR5, whereas the principal receptor for entry of T-cell line-tropic (T-tropic) strains was CXCR4. On the other hand, both M-tropic and T-tropic strains of simian immunodeficiency virus (SIV) can be mediated by CCR5, but not CXCR4 (Chew et al., J. Virol, 71:2705-2714 (1997); Marcon et al., J. Virol, 71:2522-2527 (1997); and Edinger et al., Proc. Natl. Acad. Sci. USA, 94:4005-4010 (1997)). More importantly, SIV strains were also found to infect CD⁴⁺ cells that lack CCR5 (Chen et al., 1997, supra; and Edinger et al., 1997, supra).

In humans, CCR5-tropic viruses are primarily involved in transmission, while viruses with broader tropism, particularly for CXCR4, emerge during progression to immunodeficiency (Fauci, Nature, 384:529-534 (1996)). It is not yet known whether appearance of CXCR4-tropic viruses is a consequence or the cause of immune system decline. Insight into this key problem of virus evolution is likely to require experimental manipulation in animal models. Infection of non-human primates with SIV is currently the only good animal model for studying pathogenesis of the immunodeficiency viruses (Desrosiers, Annu Rev Immunol, 8:557-578 (1990)). Moreover, different species of non-human primates vary widely in their responses to SIV infection. For example, Rhesus macaques succumb to immunodeficiency that closely resembles AIDS in humans, but sooty mangabeys and African green monkeys can sustain infection with little evidence of immune system damage (Kestler, Science, 248:1109-1112 (1990)). These interspecies differences provide important clues for understanding and combating disease progression in HIV-infected humans.

The replication of retroviruses within target cells requires the participation of host factors at every step of the virus lifecycle. Indeed, genetic screens have suggested hundreds of host factors that contribute to HIV-1 replication. In contrast, hosts have developed potent retroviral restrictive proteins, which act as an intrinsic defense mechanism. Bushman et al. PLoS Pathog (2009) 5: e1000437; Bieniasz Cell Host Microbe (2009) 5: 550-558; Strebel et al. BMC Med (2009) 7: 48.

Arguably, the most prominent of this group are the APOBEC3 proteins, which manifest a potent cellular defense mechanism that has expanded in the primate family to prevent infection by viruses that require the production of ssDNA as part of their lifecycle. Cullen J Virol (2006) 80: 1067-1076. This model of an antiviral countermeasure has been of particular importance in the quest to better understand the interaction between HIV-1 and various host proteins. Though APOBEC3G was initially identified as an inhibitor of HIV-1 replication in the absence of vif, it has since been suggested that APOBEC3G exerts a more nuanced role inside the cell and during the replication cycle of HIV-1. Sheehy et al. Nature (2002) 418: 646-650; Gallois-Montbrun et al. J. Virol (2007) 81: 2165-2178. Recent evidence suggests a complex interplay between APOBEC3G and cytoplasmic foci of protein—referred to as P-bodies—which are thought to be involved in variety of RNA processing functions. Beckham et al. (2008) Cell Host Microbe 3:206-212; Eulalio et al. Nat Rev Mol Cell Biol (2007) 8: 9-22. Esnault et al., Nature (2005) 433:430-433 report that an APOBEC3G cytidine deaminase inhibits retrotransposition of endogenous retroviruses.

One of the components of P-body complexes is a protein called Moloney Leukemia Virus 10 (Mov10), which is the dominant protein that co-immunoprecipitates with APOBEC3G complexes localized to the P-bodies Gallois-Montbrun et al. J. Virol (2007) 81:2165-2178; Kozak et al. J Biol Chem (2006) 281: 29105-29119. Mov10 was first identified in screens that examined failure of infectious Moloney murine leukemia virus (MLV) in mice Schnieke et al. J Virol (1983) 45:505-513. Subsequent sequence analysis revealed seven consensus sequences of RNA helicases at the C-terminal end of the protein Dalmay et al. Embo J (2001) 20: 2069-2078, but it is not known if these sequences have biological function or if they can bind RNA. Numerous groups have now found that Mov10 has a potent ability to perturb HIV-1 production when endogenous levels of the host protein are manipulated. Furtak et al. PLoS One 5:e9081; Burdick et al., J Virol 84:10241-10253; Wang et al., J Biol Chem 285:14346-14355. The role of Mov10 in virus production is not limited to HIV, however, as it has been shown that Mov10 can suppress numerous retroviruses Furtak et al. PLoS One 5:e9081 and is required for hepatitis D virus replication Haussecker et al. Nat Struct Mol Biol (2008) 15:714-721. Other fields have begun to implicate Mov10 as playing a critical role in protein translation control at neuronal synapses Banerjee et al. Neuron (2009) 64:871-884, a potential function as suppressor of retrotransposable elements Frost et al. Proc Natl Acad Sci USA 107:11847-11852, and even as a mediator of gene silencing El Messaoudi-Aubert et al. Nat Struct Mol Biol 17:862-868.

Some results suggest that Mov10 is both required by viruses as well as toxic to them. Transient overexpression of Mov10 in cells producing HIV-1 can reduce both the amount of virus produced as well as the per-particle infectivity of what little virus makes it out of the cell Furtak et al. PLoS One 5:e9081. In fact, this effect is exquisitely titratable. Conversely, reduction of endogenous Mov10 by siRNA-mediated knockdown similarly reduces HIV-1 production. Furtak et al. PLoS One 5:e9081, unpublished data. In fact, this viral dependence upon Mov10 extends beyond retroviruses. Haussecker, et al. showed that siRNA-mediated knockdown of Mov10 significantly reduced hepatitis D virus replication, Haussecker et al. Nat Struct Mol Biol (2008) 15:714-721, suggesting a more global role for this protein in the metabolism and processing of viral RNA. Taken together, these data indicate that endogenous levels of Mov10 must be appropriate to permit successful replication of numerous viruses.

RNA control points are important in relation to the ‘life’ cycles of viruses that employ RNA. P-bodies are critical—and their disruption crippling—for viruses such as HIV-1. (Beckham et al. Cell Host Microbe (2008) 3:206-212; Nathans et al. Mol Cell (2009)34:696-7097; Lever Adv Pharmacol (2007) 55:1-32) Mov10 is known to traffic to P-bodies Meister et al. Curr Biol (2005) 15:2149-2155, and it associates with members of the Argonaut family in the RNAi-Induced Signaling Complex (RISC) (Chendrimada et al. Nature (2007) 447:823-828) and Argonauts are themselves prominent components of P-bodies. Eulalio et al. Nat Rev Mol Cell Biol (2007) 8:9-22 APOBEC3G is also present in P-bodies. Eulalio et al. Nat Rev Mol Cell Biol (2007) 8:9-22 APOBEC3G exists in cells associated with dynamic ribonucleoprotein (RNP) complexes that variably silence or direct activity of this genome-editing protein and these complexes are not always associated with P-bodies. Gallois-Montbrun et al. J. Virol (2007) 81:2165-2178 Similarly, the Argonaut proteins responsible for assembling the RISC (especially Ago2) do not need the P-body to function (Eulalio et al. Mol Cell Biol (2007) 27:3970-3981) are diffusely distributed in the cytoplasm of somatic cells, and traffic also in stress granules—structures of protein and mRNA that are induced upon cellular stress. Leung et al. Proc Natl Acad Sci USA (2006) 103:18125-18130.

The human genome is riddled with the specters of viruses past—mostly in the form of retrotransposable genetic elements. As much as 45% of the human genome is made up of these pieces of DNA that occasionally transpose themselves after passing through an RNA intermediate. Lander et al. Nature (2001) 409:860-921; Cordaux et al. Nat Rev Genet (2009)10:691-703. If these elements are not kept under constant suppression, their replication and unabated genome-jumping would be catastrophic for the cell and, potentially, the host. Reactivation of retrotransposable elements and the related group of elements, endogenous retroviruses, has been linked to cancer (Romanish et al. Semin Cancer Biol 20:246-253; Montoya-Durango et al. Curr Mol Med 10:511-521) autoimmune disease (Balada et al. Int Rev Immunol 29:351-370) and germ cell death (Frost et al. Proc Natl Acad Sci USA 107:11847-11852). Frost et al. showed that a Mov10-like protein in mice was necessary for protecting dividing germ cells from death by retrotransposon reactivation. Frost et al. Proc Natl Acad Sci USA 107:11847-11852

Retrotransposons are typically classified as those that contain long-terminal repeats (LTRs) and those that do not. Cordaux et al. Nat Rev Genet (2009) 10:691-703. These can then be subclassified by their size and sequence similarity. In studying the activation of these elements, there are a number of plasmid constructs that contain a resistance gene interrupted by an intron and flanked by various retrotransposon signal sequences. The resistance gene only produces the appropriate protein when it is inserted into the genomic DNA. Many retrotransposons have lost their ability to code for the enzymes necessary for reverse-transcription and/or integration, and so they are only able to transpose when these enzymes are provided by other retrotransposons or endogenous retroviruses. Retrotransposon research plasmids rely on the presence of this activity for their integration and the level of integration (as measured by survival through selection) is a rough measure of retrotransposition in a cell. It is estimated that a single retroelement (Alu elements) is responsible for 0.1% of all human diseases with genetic causes. (Deininger et al., (1999) Molecular Genetics and Metabolism 67:183-93) These uncontrolled transposon insertions have also been implicated in various forms of cancer.

### SUMMARY OF THE INVENTION

The present invention is based in part upon the discovery that a Moloney Leukemia Virus 10 (Mov10) protein regulates the infectivity and replication of retroviruses such as HIV. The present invention further demonstrates that Mov10 is part of a large protein machinery called p-bodies or RNA processing bodies. The present invention is based upon the fact that HIV requires this machinery in order to effectively assemble the viral particles. Therefore, the present invention provides a novel target for intervention to limit virus spread or transmission.

In a first aspect, the present invention provides methods of inhibiting a retrovirus replication by administering a therapeutically effective amount of Mov10 or a biologically active fragment, derivative or analog thereof or of inhibiting a retrovirus replication by increasing transcription, translation or biological activity of Mov10 or a biologically active fragment, derivative or analog thereof. In some instances the retrovirus is an HIV such as, for example HIV-1. In some embodiments, the transcription, translation or biological activity of Mov10 or a biologically active fragment, derivative or analog thereof may be increased about 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample. Likewise, replication of the retrovirus may be inhibited or reduced in some instance by about 5%, 10%, 20%, 30%, 40%, 50%, 60%, 75%, 80%, 90%, 95%, 97%, 99% or even 100%.

In a second aspect, the present invention provides methods of inhibiting a retrovirus infectivity by administering a therapeutically effective amount of Mov10 or a biologically active fragment, derivative or analog thereof or of inhibiting a retrovirus replication by increasing transcription, translation or biological activity of Mov10 or a biologically active fragment, derivative or analog thereof. In some instances the retrovirus is an HIV such as, for example HIV-1. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample. Likewise, infectivity of the retrovirus may be inhibited or reduced in some instance by about 5%, 10%, 20%, 30%, 40%, 50%, 60%, 75%, 80%, 90%, 95%, 97%, 99% or even 100%.

In a third aspect, the present invention provides methods of treating a disease caused all or in part by a retrovirus, such as HIV or HIV-1, by administering a therapeutically effective amount of Mov10 or a biologically active fragment, derivative or analog thereof or by increasing transcription, translation or biological activity of Mov10 or a biologically active fragment, derivative or analog thereof. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

In an fourth aspect, the present invention provides methods to identify agents such as small molecules, proteins and antibodies, that may inhibit a retrovirus by identifying an agent that is regulated by Mov10 or a biologically active fragment, derivative or analog thereof. Such an agent that is regulated by Mov10 or a biologically active fragment, derivative or analog thereof may in some instances be overexpressed, underexpressed, upregulated or downregulated in response to Mov10 overexpression, underexpression, enhanced biological activity or reduced biological activity. Similarly, the present invention also provides methods to identify targets that are regulated by Mov10 or a biologically active fragment, derivative or analog thereof that can impact HIV or other retrovirus processing. Such methods feature administering Mov10 or increasing or decreasing the biological activity of Mov10 in a biological sample or in an organism and then identifying one or more agents such as, for example, a protein or a peptide whose expression, concentration or biological activity is either increased or decreased as a result of administering Mov10 or increasing or decreasing the biological activity of Mov10 in the biological sample or in the organism. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

This aspect of the invention provides a novel target that can be manipulated to inhibit a retrovirus replication or infectivity or treat a disease caused all or in part by a retrovirus. Agents such as small molecules, proteins and antibodies may be identified by standard assay techniques known in the art as applied to identify those agents that increase or decrease the biological activity or expression of Mov10 or a biologically active fragment, derivative or analog thereof. Agents so identified may be useful to treat a disease that may be successfully treated, all or in part, by modulating biological activity of Mov10 or a biologically active fragment, derivative or analog thereof. The disease that may be successfully treated, all or in part, may be, for instance, a retroviral infection. As such, these methods are also methods of screening for therapeutic agents effective to treat a disease caused all or in part by a retrovirus infection.

In a fifth aspect, the present invention provides methods to monitor or assess a retrovirus infection, transmission or spread, such as, for example, HIV or HIV-1 infection, transmission or spread by monitoring increased or decreased expression levels of Mov10, increased or decreased biological activity of Mov10, or increased or decreased expression or biological activity of one or more other p-body components. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

In a sixth aspect, the present invention provides pharmaceutical compositions comprising a therapeutically effective amount of Mov10 or a biologically active fragment, derivative or analog thereof in combination with a pharmaceutically acceptable carrier. Such a pharmaceutical composition may be useful for decreasing replication of a retrovirus, such as HIV or HIV-1, decreasing infectivity of a retrovirus, such as HIV or HIV-1, or treating a disease caused all or in part by a retrovirus, such as HIV or HIV-1.

In a seventh aspect, the present invention provides pharmaceutical compositions comprising a therapeutically effective amount of a Mov10 inhibitor, such as, for instance an antibody or a nucleic acid that reduces transcription of the Mov10 gene or translation of a Mov10 protein or a biologically active fragment, derivative or analog thereof in combination with a pharmaceutically acceptable carrier. Such a pharmaceutical composition may be useful for decreasing replication of a retrovirus, such as HIV or HIV-1, decreasing infectivity of a retrovirus, such as HIV or HIV-1, or treating a disease caused all or in part by a retrovirus, such as HIV or HIV-1.

In an eighth aspect, the present invention provides methods of inhibiting a retrovirus replication by administering a therapeutically effective amount of a Mov10 inhibitor, such as, for instance an antibody or a nucleic acid that reduces transcription of the Mov10 gene or translation of a Mov10 protein or a biologically active fragment, derivative or analog thereof In some instances the retrovirus is an HIV such as, for example HIV-1. In some embodiments, the transcription, translation or biological activity of Mov10 or a biologically active fragment, derivative or analog thereof may be decreased about 5%, 10%, 20%, 30%, 40%, 50%, 60%, 75%, 80%, 90%, two times, three times, four times, five times, ten times, twenty times, or even fifty or a hundred times less than the transcription, translation or biological activity of Mov10 or a biologically active fragment, derivative or analog thereof in a wild type cell or biological sample. Likewise, replication of the retrovirus may be inhibited or reduced in some instance by about 5%, 10%, 20%, 30%, 40%, 50%, 60%, 75%, 80%, 90%, 95%, 97%, 99% or even 100%.

In a ninth aspect, the present invention provides methods of inhibiting a retrovirus infectivity by administering a therapeutically effective amount of a Mov10 inhibitor, such as, for instance an antibody or a nucleic acid that reduces transcription of the Mov10 gene or translation of a Mov10 protein or a biologically active fragment, derivative or analog thereof or of inhibiting a retrovirus infectivity by decreasing transcription, translation or biological activity of Mov10 or a biologically active fragment, derivative or analog thereof. In some instances the retrovirus is an HIV such as, for example HIV-1. In some embodiments, the transcription, translation or biological activity of Mov10 or a biologically active fragment, derivative or analog thereof may be decreased by about 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample. Likewise, infectivity of the retrovirus may be inhibited or reduced in some instance by about 5%, 10%, 20%, 30%, 40%, 50%, 60%, 75%, 80%, 90%, 95%, 97%, 99% or even 100%.

In a tenth aspect, the present invention provides methods of treating a disease caused all or in part by a retrovirus, such as HIV or HIV-1, by administering a therapeutically effective amount of a Mov10 inhibitor, such as, for instance an antibody or a nucleic acid that reduces transcription of the Mov10 gene or translation of a Mov10 protein or a biologically active fragment, derivative or analog thereof. In some embodiments, the transcription, translation or biological activity of Mov10 or a biologically active fragment, derivative or analog thereof may be decreased by about 5%, 10%, 20%, 30%, 40%, 50%, 60%, 75%, 80%, 90%, 95%, 97%, 99% or even 100%.

In an eleventh aspect, the present invention provides methods of inhibiting efficiency of retrovirus incorporation into a cellular genome or retrotransposition by administering a p-body protein or a biologically active fragment, derivative or analog thereof or by increasing transcription, translation or biological activity of a p-body protein or a biologically active fragment, derivative or analog thereof. In some embodiments, the transcription, translation or biological activity of the p-body protein or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of the p-body protein or a biologically active fragment, derivative or analog thereof in a wild type cell or biological sample. The p-body protein may be in some instances PIWI, Apobec3A, Rent1, Mov10 or Ago2. Similarly, this aspect of the invention provides methods for treating or preventing a genetic disease, methods for reducing the risk of cancer, methods of treating cancer, and methods for preventing cancer.

### BRIEF DESCRIPTION OF THE FIGURES

FIG. 1 demonstrates that overexpression of Mov10 decreases HIV-1 infectivity. (A) 293T cells were transfected with different amounts of Mov10 plasmid, and the expression of Mov10 was determined by Western blot. (B) 293T cells were transfected with 0.5 μg of either pCMV6-XL5 plasmid (control), Mov10 or APOBEC3G in the presence or absence of 0.5 μg vif as well as 1 μg HIV-1-GFP (Δenv, Δvif, Δvpr, Δnef) and 0.5 μg p-L-VSV-G. Virus was collected 24 h later, and then added to Jurkat T cells. Virus transfer was standardized across treatment conditions by p24 levels as described (Materials and Methods). Percent infected cells was then determined using FACS analysis for GFP-expression after virus was allowed to incubate with target cells for 72 hours. Error bars represent one standard deviation. Supernatants of 293T cells that had been transfected with varying amounts of Mov10-expressing plasmid were assayed for (C) HIV-1 CA (p24) levels and (D) infectivity after standardization by p24 content. The particular HIV-1 vector used in this experiment expresses GFP and lacks any of the HIV accessory genes (Vif, Vpr, Nef and Vpu). For simplicity, the amount of Mov10 plasmid that was transfected is expressed as a ratio of HIV-1 plasmid to Mov10 plasmid and plotted logarithmically. Mov10 plasmid was used at levels of 1/6 to 1/1500 that of the HIV-1 plasmid. Error bars represent one standard deviation.

FIG. 2 represents that Mov10 decreases specific infectivity of HIV-1. Supernatants of 293T cells that had been transfected with varying amounts of Mov10-expressing plasmid were assayed for (A) HIV-1.Luc p24 levels and (B) after standardization by p24 content, infectivity of target cells was determined by luciferase activity from VSVG.HIV.Luc, which encodes all the HIV accessory genes. For simplicity, the amount of Mov10 plasmid that was transfected is expressed as a ratio of HIV-1 plasmid to Mov10 plasmid and plotted logarithmically. In the experiment (see Materials and Methods for further explanation) HIV-1 plasmid levels remained constant, while Mov10 plasmid was used at levels of 1/6 to 1/1500 that of the HIV-1 plasmid. Error bars represent one standard deviation. (C) Representative plots of Jurkat T cells infected with GFP-expressing virus produced in the presence of either pcDNA3 or Mov10 (ratio of Mov10 to HIV-1 plasmid was 1/25) three days after infection. Quantification of experiments performed using GFP-expressing virus is shown in supplemental FIG. 1. (D) HIV-1 produced in cells stably expressing Mov10 is less infectious. 293T cells were stably transfected with a vector expressing Mov10 and selected with 0.5 mg/ml G418. When these cells were subsequently transfected with an HIV-1 vector, the virus produced from them was less infectious to Jurkat T cells than virus produced in cells stably transfected with a control (pcDNA3) vector. Error bars represent standard error of the mean.

FIG. 3 depicts that Mov10 impairs HIV-1 replication in primary CD4+ T cells. Supernatants of activated CD4⁺ cells that had been nucleofected with a replication competent, CCR5-tropic HIV-1 viral plasmid and either Mov10 or control (pcDNA3) plasmid ((A) schematic) were assayed for p24 production (B) and then standardized by p24 concentration and used to infect CCR5⁺ Hut cells. (C) Infection success was determined by flow cytometry analysis of GFP expression.

FIG. 4 depicts broad inhibition of infectious retroviruses by Mov10. Virions derived from 293T cells transfected with various viral plasmids (as described in Materials and Methods) and either pcDNA3 or pcDNA3-Mov10 were used to infect HeLa cells. The % infected cells represents the percentage of GFP-positive cells in the cell population.

FIG. 5 demonstrates that an optimal concentration of Mov10 is required for HIV-1 infectivity. 293T cells were transfected with a non-targeting siRNA or Mov10-specific siRNA. At 48 h post-siRNA transfection, the cells were transfected with 1.5 μg of pHIV-RFP, 0.7 μg of p-L-VSV-G and increasing amounts of the Mov10 expression plasmid. At 96 h post-siRNA transfection: (A) the cell lysates were examined by Western blot for Mov10 levels using an anti-Mov10 antibody. Equal protein loading was confirmed by probing with anti-tubulin antibody. (B) After normalizing for p24 values, virus obtained from the transfections was used to infect HeLa cells and infectivity was measured by FACS. The % infected cells represents the percentage of RFP-positive cells in the cell population. Error bars represent one standard deviation.

FIG. 6 demonstrates that viral glycoprotein incorporation is not affected by Mov10 overexpression. 293T cells were transfected with plasmids necessary for the production of VSV-G pseudotyped Virion-Like Particles (VLPs) containing a GFP tag (as a Gag-GFP fusion) as well as either control or Mov10. Supernatant from these cells was then collected and added to Jurkat T cells and assayed for binding to target cells. Cells bound by one or more VLPs are GFP⁺ by FACS.

FIG. 7 demonstrates that early and late reverse transcription is suppressed in virus produced from cells overexpressing Mov10. Synthesis of reverse transcripts by real-time PCR after infection by HIV-1 produced in the presence of either empty vector (pcDNA3) or Mov10 expressing plasmid was measured. No significant difference between production of (A) Early (R-U5) or (B) Late (R-Gag) DNA products of reverse transcription was observed. (C) Infectivity of virions produced in the presence of Mov10 was significantly inhibited. Input viruses were normalized for p24. Results are representative of one out of three similar experiments.

FIG. 8 demonstrates that the helicase domain of Mov10 is not required for HIV-1 restriction. Virions were produced by cotransfection of 293T cells with HIV-1 vector and either empty vector or Mov10, Mov10 N-terminus, Mov10 C-terminus or the putative helicase motif mutant expression plasmids. (A) Schematic of wild-type and mutated human Mov10 constructs. (B) Cell lysates were probed with anti-HA, anti-Mov10, and anti-tubulin. Lanes: 1) pcDNA3, 2) Mov10, 3) Mov10 Nterm, 4) Mov10 Cterm, and 5) Mov10-EQ. Arrows indicate molecular weight of native Mov10. (C) Infectivity of virions produced was examined by FACS after infection of HeLa cells. The % infectivity represents the percentage of RFP-positive cells in the cell population. Error bars represent one standard deviation.

FIG. 9 demonstrates that Mov10 suppresses the activity of LTR and non-LTR retrotransposons. HeLa cells were co-transfected with a retrotransposon construct and Mov10 in ratios of 3:1, 15:1, 30:1, or 750:1 (mass construct:mass Mov10 plasmid) and then reseeded in G418 selection media after 4 days. Only plasmids that were successfully reverse-transcribed and integrated into the genome could allow a cell to survive selection. Cells were subsequently expanded for 14 days under selection and their colonies counted to determine efficiency of retrotransposition. Baseline retrotransposition in cells transfected with only the appropriate retrotransposon construct and a pCMV6 empty vector control is normalized to 100% for each construct. The retrotransposon constructs express the endogenous retroviruses (LTR-containing) IAP (Dewannieux et al., (2004) Nat Genet 36: 534-539), or MusD (Ribet et al., (2004) Genome Res 14: 2261-2267); or the non-LTR Line1 construct (Cordaux et al., (2009) Nat Rev Genet 10: 691-703). Error bars represent standard deviation of experiments run in duplicate.

FIG. 10 provides the Mov10 gene in genomic location with bands according to Ensembl, locations according to Gene Loc. Mov10 is a RNA helicase required for RNA-mediated gene silencing by the RNA-induced silencing complex (RISC). It is required for both miRNA-mediated translational repression and miRNA-mediated cleavage of complementary mRNAs by RISC. It is also required for RNA-directed transcription and replication of the human hapatitis delta virus (HDV). It Interacts with small capped HDV RNAs derived from genomic hairpin structures that mark the initiation sites of RNA-dependent HDV RNA transcription.

FIG. 11 demonstrates dose dependent inhibition of retrotransposon by Mov10. A) Western blot of Mov10 preparations of transfected HeLa cells. B) Analysis of activity of Mov10 on different retroelements defined by G418R colonies. Retrotranspositions are percentages relative to control (empty plasmid). Data are means±s.d. of 2 independent experiments.

FIG. 12 demonstrates the effect of different p-pody proteins on retrotransposition. Retrotranspositions are percentages relative to control (empty plasmid). Data are means±s.d. of 2 independent experiments.

### DETAILED DESCRIPTION OF THE INVENTION

The present invention demonstrates that Mov10 protein can regulate the replication and infectivity of HIV and other retroviruses. The present invention further demonstrates that Mov10 is part of a large protein machinery called p-bodies or RNA processing bodies. The present invention is based upon the fact that HIV requires this machinery in order to effectively assemble the viral particles. Therefore, the present invention provides a novel target for intervention to limit virus spread or transmission.

In a first aspect, the present invention provides methods of inhibiting a retrovirus replication by administering a therapeutically effective amount of Mov10 or a biologically active fragment, derivative or analog thereof or of inhibiting a retrovirus replication by increasing transcription, translation or biological activity of Mov10 or a biologically active fragment, derivative or analog thereof. In some instances the retrovirus is an HIV such as, for example HIV-1. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

In a second aspect, the present invention provides methods of inhibiting a retrovirus infectivity by administering a therapeutically effective amount of Mov10 or a biologically active fragment, derivative or analog thereof or of inhibiting a retrovirus replication by increasing transcription, translation or biological activity of Mov10 or a biologically active fragment, derivative or analog thereof. In some instances the retrovirus is an HIV such as, for example HIV-1. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

In a third aspect, the present invention provides methods of treating a disease caused all or in part by a retrovirus, such as HIV or HIV-1, by administering a therapeutically effective amount of Mov10 or a biologically active fragment, derivative or analog thereof or by increasing transcription, translation or biological activity of Mov10 or a biologically active fragment, derivative or analog thereof. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

In an fourth aspect, the present invention provides methods to identify agents such as small molecules, proteins and antibodies, that may inhibit a retrovirus by identifying an agent that is regulated by Mov10 or a biologically active fragment, derivative or analog thereof. Such an agent that is regulated by Mov10 or a biologically active fragment, derivative or analog thereof may in some instances be overexpressed, underexpressed, upregulated or downregulated in response to Mov10 overexpression, underexpression, enhanced biological activity or reduced biological activity. Similarly, the present invention also provides methods to identify targets that are regulated by Mov10 or a biologically active fragment, derivative or analog thereof that can impact HIV or other retrovirus processing. Such methods feature administering Mov10 or increasing or decreasing the biological activity of Mov10 in a biological sample or in an organism and then identifying one or more agents such as, for example, a protein or a peptide whose expression, concentration or biological activity is either increased or decreased as a result of administering Mov10 or increasing or decreasing the biological activity of Mov10 in the biological sample or in the organism. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

This aspect of the invention provides a novel target that can be manipulated to inhibit a retrovirus replication or infectivity or treat a disease caused all or in part by a retrovirus. Agents such as small molecules, proteins and antibodies may be identified by standard assay techniques known in the art as applied to identify those agents that increase or decrease the biological activity or expression of Mov10 or a biologically active fragment, derivative or analog thereof. Agents so identified may be useful to treat a disease that may be successfully treated, all or in part, by modulating biological activity of Mov10 or a biologically active fragment, derivative or analog thereof. The disease that may be successfully treated, all or in part, may be, for instance, a retroviral infection. As such, these methods are also methods of screening for therapeutic agents effective to treat a disease caused all or in part by a retrovirus infection.

In a fifth aspect, the present invention provides methods to monitor or assess a retrovirus infection, transmission or spread, such as, for example, HIV or HIV-1 infection, transmission or spread by monitoring increased or decreased expression levels of Mov10, increased or decreased biological activity of Mov10, or increased or decreased expression or biological activity of one or more other p-body components. In some embodiments, the transcription, translation or biological activity of Mov10 or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample.

In a sixth aspect, the present invention provides pharmaceutical compositions comprising a therapeutically effective amount of Mov10 or a biologically active fragment, derivative or analog thereof in combination with a pharmaceutically acceptable carrier. Such a pharmaceutical composition may be useful for decreasing replication of a retrovirus, such as HIV or HIV-1, decreasing infectivity of a retrovirus, such as HIV or HIV-1, or treating a disease caused all or in part by a retrovirus, such as HIV or HIV-1.

In a seventh aspect, the present invention provides pharmaceutical compositions comprising a therapeutically effective amount of a Mov10 inhibitor, such as, for instance an antibody or a nucleic acid that reduces transcription of the Mov10 gene or translation of a Mov10 protein or a biologically active fragment, derivative or analog thereof in combination with a pharmaceutically acceptable carrier. Such a pharmaceutical composition may be useful for decreasing replication of a retrovirus, such as HIV or HIV-1, decreasing infectivity of a retrovirus, such as HIV or HIV-1, or treating a disease caused all or in part by a retrovirus, such as HIV or HIV-1.

In an eighth aspect, the present invention provides methods of inhibiting a retrovirus replication by administering a therapeutically effective amount of a Mov10 inhibitor, such as, for instance an antibody or a nucleic acid that reduces transcription of the Mov10 gene or translation of a Mov10 protein or a biologically active fragment, derivative or analog thereof In some instances the retrovirus is an HIV such as, for example HIV-1. In some embodiments, the transcription, translation or biological activity of Mov10 or a biologically active fragment, derivative or analog thereof may be decreased about 5%, 10%, 20%, 30%, 40%, 50%, 60%, 75%, 80%, 90%, two times, three times, four times, five times, ten times, twenty times, or even fifty or a hundred times less than the transcription, translation or biological activity of Mov10 or a biologically active fragment, derivative or analog thereof in a wild type cell or biological sample. Likewise, replication of the retrovirus may be inhibited or reduced in some instance by about 5%, 10%, 20%, 30%, 40%, 50%, 60%, 75%, 80%, 90%, 95%, 97%, 99% or even 100%.

In a ninth aspect, the present invention provides methods of inhibiting a retrovirus infectivity by administering a therapeutically effective amount of a Mov10 inhibitor, such as, for instance an antibody or a nucleic acid that reduces transcription of the Mov10 gene or translation of a Mov10 protein or a biologically active fragment, derivative or analog thereof or of inhibiting a retrovirus infectivity by decreasing transcription, translation or biological activity of Mov10 or a biologically active fragment, derivative or analog thereof. In some instances the retrovirus is an HIV such as, for example HIV-1. In some embodiments, the transcription, translation or biological activity of Mov10 or a biologically active fragment, derivative or analog thereof may be decreased by about 5%, 10%, 20%, 30%, 40%, 50%, 60%, 75%, 80%, 90%, two times, three times, four times, five times, ten times, twenty times, or even fifty or a hundred times more than the transcription, translation or biological activity of Mov10 or a biologically active fragment, derivative or analog thereof in a wild type cell or biological sample. Likewise, infectivity of the retrovirus may be inhibited or reduced in some instance by about 5%, 10%, 20%, 30%, 40%, 50%, 60%, 75%, 80%, 90%, 95%, 97%, 99% or even 100%.

In a tenth aspect, the present invention provides methods of treating a disease caused all or in part by a retrovirus, such as HIV or HIV-1, by administering a therapeutically effective amount of a Mov10 inhibitor, such as, for instance an antibody or a nucleic acid that reduces transcription of the Mov10 gene or translation of a Mov10 protein or a biologically active fragment, derivative or analog thereof. In some embodiments, the transcription, translation or biological activity of Mov10 or a biologically active fragment, derivative or analog thereof may be decreased by about 5%, 10%, 20%, 30%, 40%, 50%, 60%, 75%, 80%, 90%, 95%, 97%, 99% or even 100%.

In an eleventh aspect, the present invention provides methods of inhibiting efficiency of retrovirus incorporation into a cellular genome or retrotransposition by administering a p-body protein or a biologically active fragment, derivative or analog thereof or by increasing transcription, translation or biological activity of a p-body protein or a biologically active fragment, derivative or analog thereof. In some embodiments, the transcription, translation or biological activity of the p-body protein or a biologically active fragment, derivative or analog thereof may be increased 5%, 10%, 20%, 30%, 40%, 50%, 60%, 75%, 80%, 90%, two times, three times, four times, five times, ten times, twenty times, or even fifty or a hundred times more than the transcription, translation or biological activity of the p-body protein or a biologically active fragment, derivative or analog thereof in a wild type cell or biological sample. The p-body protein may be in some instances PIWI, Apobec3A, Rent1, Mov10 or Ago2. Similarly, this aspect of the invention provides methods for treating or preventing a genetic disease, methods for reducing the risk of cancer, methods for treating cancer, and methods for preventing cancer.

In another aspect, the present invention provides methods to identify proteins that may inhibit HIV infection by limiting the late stage of its life cycle. As opposed to inhibition of HIV infection of HIV APOBEC3G, the virus cannot overcome the suppressive effects of Mov10 by any of the viral gene products including the Vif protein, which is highly effective in neutralizing the effects of APOBEC3G. The invention identifies a novel target that can be manipulated to regulate HIV infection.

In another aspect, the present invention provides reagents to identify mechanisms required for HIV assembly through p-bodies. The present invention also provides methods to identify additional targets that are regulated Mov10 that can impact HIV or other retrovirus processing.

In another aspect, the present invention provides methods to identify specific biological or chemical inducers of Mov10 or other p-body components. In still other aspects, the present invention provides methods to screen for chemicals that can target Mov10-virus interaction or specifically interfere with viral RNA or assembly within the p-bodies or RNA processing machinery. Additionally, the present invention provides methods to monitor or assess HIV transmission or spread using SNPs or expression levels of Mov10 and other p-body components.

The present invention also includes assays for selecting for a suspected therapeutic agent for possible use in the treatment of AIDS with the use of one of the cells of the present invention. In one particular embodiment the cell is a mammalian cell that expresses human CD4. One such method for selecting a suspected therapeutic agent for possible use in the treatment of AIDS comprises administering a potential therapeutic agent to the cell.

The present invention also includes an assay for selecting a plausible therapeutic agent for possible use in the treatment of a disease caused all or in part by a retrovirus, such as, for instance, AIDS. One such method comprises administering a suspected therapeutic agent to a transgenic non-human mammal infected with a retrovirus such as HIV.

The present invention also provides the agents obtained by such methods. The agent may be, for instance, a small organic molecule. These and other aspects of the present invention will be better appreciated by reference to the following drawings and Detailed Description. In a specific embodiment, the term “about” means within 20%, preferably within 10%, and more preferably within 5%.

When expression of Mov10 is silenced or knocked out, HIV infectivity is not only improved but in fact slightly reduced, in some instances about two fold. When Mov10 is overexpressed in 293T cells, HIV infectivity (the output of HIV that is infectious) is significantly suppressed. Thus if Mov10 was a classical restriction factor, higher levels of infectious HIV would occur when Mov10 expression is silenced. Therefore, Mov10 appears not to be a restriction factor for HIV per se. Rather, increasing (or partly decreasing) its levels in cells perturbs the p-body or other complexes in the cells that are required for viral RNA processing. Compounds (small molecules, proteins, peptides, biologicals, antibodies, etc.) that can increase and also decrease Mov10 expression are useful for suppressing HIV infection.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

As used herein, the following terms are defined as follows:

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10, amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

As used herein a “small organic molecule” is an organic compound [or organic compound complexed with an inorganic compound (e.g., metal)] that has a molecular weight of less than 3 kilodaltons, and preferably less than 1.5 kilodaltons. An “agent” of the present invention is preferably a small organic molecule.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host.

In a specific embodiment, the term “about” means within 20%, preferably within 10%, and more preferably within 5%.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding Mov10 or peptide sequences therein or comprising or consisting of sequences which are degenerate thereto. DNA sequences having the nucleic acid sequence encoding the peptides of the invention are contemplated, including degenerate sequences thereof encoding the same, or a conserved or substantially similar, amino acid sequence. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or |
| | AGC |
| Proline (Pro or P) | CCU or CCC or CCA or CCG |
| Threonine (Thr or T) | ACU or ACC or ACA or ACG |
| Alanine (Ala or A) | GCU or GCC or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Mutations can be made in the sequences encoding the protein or peptide sequences of the MMP-2 proteins, peptides or immune activator proteins or peptides of the invention, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups
Alanine, Valine, Leucine, Isoleucine, Proline, Phenylalanine, Tryptophan, Methionine
   Amino Acids with Uncharged Polar R Groups
Glycine, Serine, Threonine, Cysteine, Tyrosine, Asparagine, Glutamine
   Amino Acids with Charged Polar R Groups (Negatively Charged at Ph 6.0)
Aspartic acid, Glutamic acid

### Basic Amino Acids (Positively Charged at pH 6.0)

Lysine, Arginine, Histidine (at pH 6.0)

Another grouping may be those amino acids with phenyl groups:
Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues, preferably at least about 80%, and most preferably at least about 90, 91, 92, 93, 94, 95, 96, 97, 98, or 99% of the amino acid residues are identical, or represent conservative substitutions.

Analogs and derivatives of a protein are normally said to be substantially homologous.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

The terms “a fragment, derivative or analog thereof” refer in some instances to amino acid sequences, peptides and proteins having about 50%, 60%, 70%, 75%, 80%, 85%, 90%, 95%, 96%, 97%, 98%, 99% or about 100% identical sequence to the naturally occurring wild type Mov10 protein, such as, for instance, the Mov10 protein sequence provided in SEQ ID NO:1.

The term ‘agent’ means any molecule, including polypeptides, antibodies, polynucleotides, chemical compounds and small molecules. In particular the term agent includes compounds such as test compounds or drug candidate compounds.

The term ‘agonist’ refers to a ligand that stimulates the receptor the ligand binds to in the broadest sense or stimulates a response that would be elicited on binding of a natural ligand to a binding site.

The terms “inhibitor” or “antagonist” refers in some instances to a ligand that stimulates the receptor the ligand binds to in the broadest sense or stimulates a response that would be elicited on binding of a natural ligand to a binding site in instances where the response that is elicited results in reducing or inhibiting the biological activity of its target. The terms “inhibitor” or “antagonist” are intended to encompass agents or molecules that reduce or inhibit the biological activity of another target molecule such as a protein. Such agents or molecules may function by binding to a target molecule such as a protein or may function by reducing the amount of the target molecule such as a protein that is transcribed, translated or expressed. Such agents may be, for instance, small molecules, antibodies or nucleic acids such as, for instance, siRNA, iRNA, etc.

The term ‘assay’ means any process used to measure a specific property of a compound or agent. A ‘screening assay’ means a process used to characterize or select compounds based upon their activity from a collection of compounds.

“Preventing” or “prevention” refers to a reduction in risk of acquiring a disease or disorder.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

The term “retrovirus” includes, for instance, the following genera: Genus Alpharetrovirus; type species: Avian leukosis virus; others include Rous sarcoma virus, Genus Betaretrovirus; type species: Mouse mammary tumour virus, Genus Gammaretrovirus; type species: Murine leukemia virus; others include Feline leukemia virus, Genus Deltaretrovirus; type species: Bovine leukemia virus; others include the cancer-causing Human T-lymphotropic virus, Genus Epsilonretrovirus; type species: Walleye dermal sarcoma virus, Genus Lentivirus; type species: Human immunodeficiency virus 1; others include Simian, Feline immunodeficiency viruses, Genus Spumavirus; type species: Simian foamy virus, endogenous retroviruses such as those, from genera class I most similar to the gammaretroviruses, class II most similar to the betaretroviruses and alpharetroviruses, and class III most similar to the spumaviruses, and group VI retroviruses that use virally encoded reverse transcriptase, an RNA-dependent DNA polymerase, to produce DNA from the initial virion RNA genome. This DNA is often integrated into the host genome, as in the case of retroviruses and pseudoviruses, where it is replicated and transcribed by the host. Group VI includes, for instance, family Metaviridae, family Pseudoviridae, family Retroviridae—Retroviruses, e.g. HIV, Group VII that have DNA genomes contained within the invading virus particles. The DNA genome is transcribed into both mRNA, for use as a transcript in protein synthesis, and pre-genomic RNA, for use as the template during genome replication. Virally encoded reverse transcriptase uses the pre-genomic RNA as a template for the creation of genomic DNA. Group VII includes, for instance family Hepadnaviridae, e.g. Hepatitis B virus and family Caulimoviridae,—e.g. Cauliflower mosaic virus.

By “infectivity” is meant the relative likelihood of an uninfected cell or individual becoming infected upon contact or close encounter with an infected cell or individual. “Infectivity” may be proportional to the relative or absolute numbers of infectious organisms present in the infected cell or individual. As such, reducing, inhibiting or decreasing infectivity of a virus may include reducing the number of or the rate at which new cells become infected with the virus.

By “replication” of a virus such as a retrovirus is meant the process of intracellular viral multiplication, consisting of the synthesis of proteins, nucleic acids, and sometimes lipids, and their assembly into a new infectious particle. As such, reducing, inhibiting or decreasing replication of a virus may include reducing the rate or speed at which synthesis of one or more proteins, nucleic acids, or lipids, and their assembly into a new infectious particle occurs.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

The Mov10 protein sequence (SEQ ID NO: 1) is provided in Table 1.

### Table 1

1 mpskfscrql reagqcfesf lvvrgldmet drerlrtiyn rdfkisfgtp apgfssmlyg

61 mkianlayvt ktrvrffrld rwadvrfpek rrmklgsdis khhksllaki fydraeylhg

121 khgvdvevqg pheardgqll irldlnrkev ltlrlrnggt qsvtlthlfp lcrtpqfafy

181 nedqelpcpl gpgecyelhv hcktsfvgyf patvlwellg pgesgsegag tfyiarflaa

241 vahsplaaql kpmtpfkrtr itgnpvvtnr ieegerpdra kgydlelsma lgtyyppprl

301 rqllpmllqg tsiftapkei aeikaqleta lkwrnyevkl rlllhleelq mehdirhydl

361 esvpmtwdpv dqnprlltle vpgvtesrps vlrgdhlfal lssethqedp itykgfvhkv

421 eldrvklsfs msllsrfvdg ltfkvnftfn rqplrvqhra leltgrwllw pmlfpvaprd

481 vpllpsdvkl klydrslesn peqlqamrhi vtgttrpapy iifgppgtgk tvtiveaikq

541 vvkhlpkahi lacapsnsga dllcqrlrvh lpssiyrlla psrdirmvpe dikpccnwda

601 kkgeyvfpak kklgeyrvli ttlitagrlv saqfpidhft hifideaghc mepeslvaia

661 glmevketgd pggqlvlagd prqlgpvlrs pltqkhglgy sllerlltyn slykkgpdgy

721 dpqfitkllr nyrshptild ipnqlyyege lqacadvvdr erfcrwaglp rqgfpiifhg

781 vmgkderegn spsffnpeea atvtsylkll lapsskkgka rlsprsvgvi spyrkqveki

841 rycitkldre lrglddikdl kvgsveefqg qersvilist vrssqsfvql dldfnlgflk

901 npkrfnvavt rakalliivg nplllghdpd wkvflefcke nggytgcpfp akldlqqgqn

961 llqglsklsp stsgphshdy 1pqeregegg lslqvepewr nel

### Overexpression of Mov10 Reduces the Specific Infectivity of HIV-1.

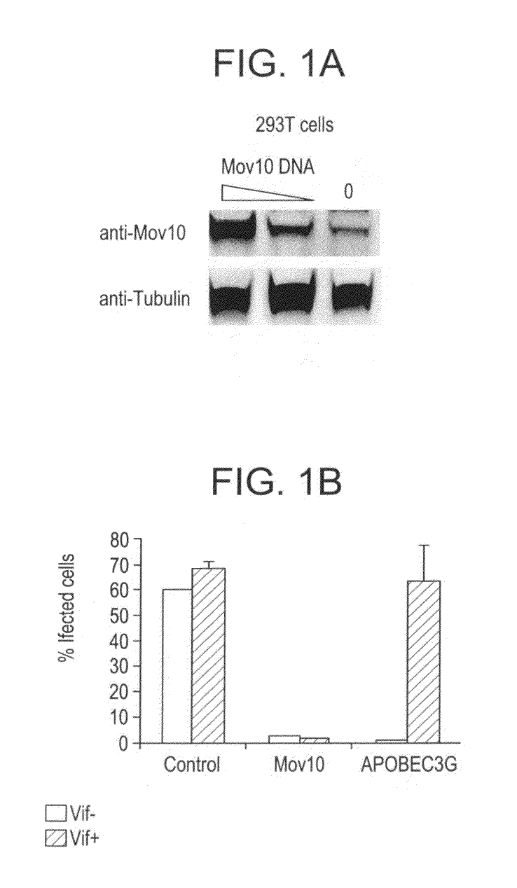

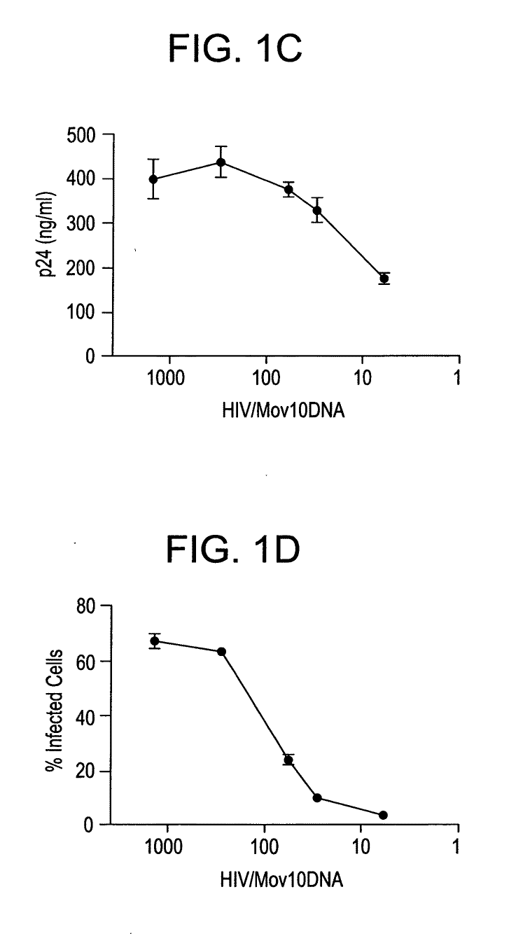

In order to identify proteins that interact with APOBEC3G, a mass spectrometry analysis of proteins co-immunoprecipitated with APOBEC3G from primary CD4⁺ T cells or 293T cells was performed. It was revealed that the putative RNA helicase, Mov10, was reproducibly and predominantly associated with high-molecular weight APOBEC3G preparations. This finding was in line with observations from other reports that detected Mov10 in similar mass spectrometry analysis (Gallois-Montbrun et al., (2007) J Virol 81: 2165-2178; Kozak et al., (2006) J Biol Chem 281: 29105-29119). Although in subsequent analysis a direct association between APOBEC3G and Mov10 (data not shown) was not found. Whether perturbation of Mov10 expression could impact HIV-1 infectivity in a manner similar to APOBEC3G was evaluated. Accordingly, 293T cells were transfected with different Mov10 plasmid amounts. Mov10 was highly expressed upon transfection (FIG. 1A), and this expression level was not immediately toxic to cells as no cell death was observed over three days following transfection (data not shown).

Whether viruses produced in the presence of ectopic Mov10 exhibited reduced infectivity similar to that typically observed with APOBEC3G-transfected 293T cells was determined. To address this point, 293T cells were co-transfected with GFP-expressing HIV-1 (with or without Vif), a vesicular somatitis virus glycoprotein (VSV-G) expression construct, and either a Mov10 or APOBEC3G expression plasmid. Two days later, VSV-G pseudotyped HIV-1 vectors produced from the transfected 293T cells were used to infect the Jurkat T cell line. Remarkably, overexpression of Mov10 almost completely abolished infectivity of HIV-1 produced by 293T cells (FIG. 1B). However, in contrast to APOBEC3G overexpression, the Mov10-mediated reduction of HIV-1 infectivity could not be rescued by co-expression of HIV-1 vif (FIG. 1B). These data demonstrate that increased levels of Mov10 have a profound effect on the generation of infectious HIV-1.

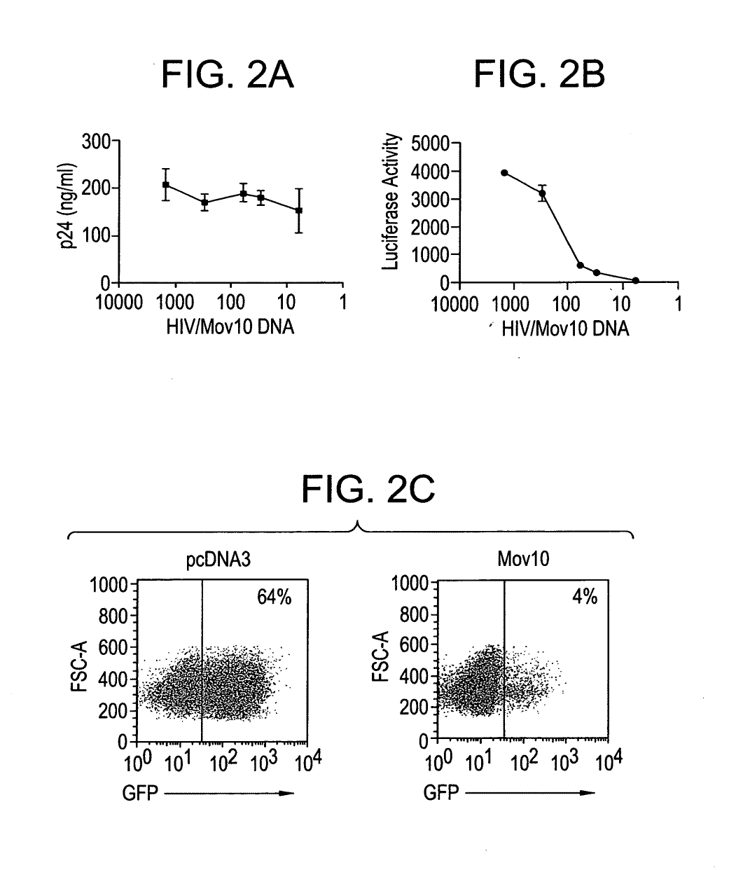

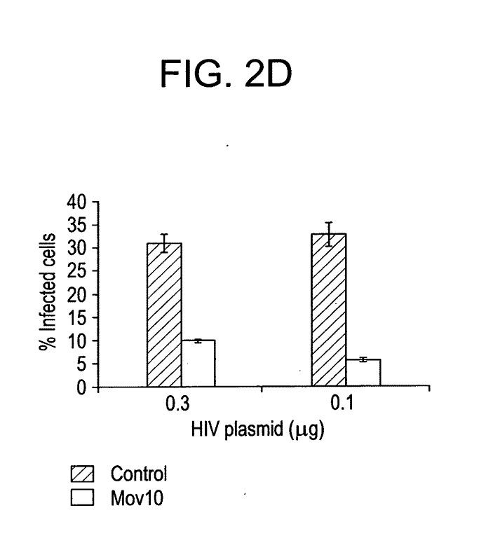

Whether the suppression of HIV-1 infectivity through Mov10 overexpression was due to reduced HIV-1 particle production was investigated. It was found that at varying ratios of plasmids encoding HIV-1 to Mov10, the HIV-1 particle production, as assessed by p24 protein levels in supernatant, was mostly unaffected (FIG. 2A and FIG. 1C) while the infectivity of the viruses normalized to p24 levels were greatly reduced using either luciferase- or GFP-expressing viruses (FIG. 2B, 2C and FIG. 1D). However, at the ratios where Mov10 levels were highest, a notable reduction in the level of p24 generated by producer cells (FIG. 2A and FIG. 1C, lowest HIV-1/Mov10 expressing plasmid ratios) was observed. Similar inhibition of HIV-1 infectivity was observed when viruses were produced from 293T cells stably overexpressing Mov10 for prolonged periods (FIG. 2D).

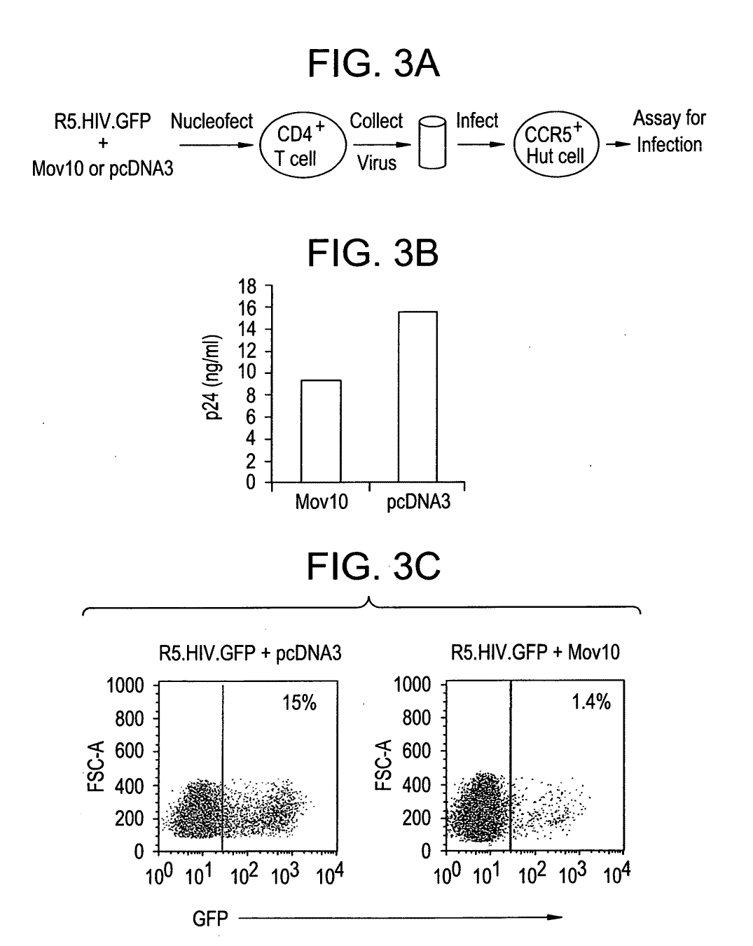

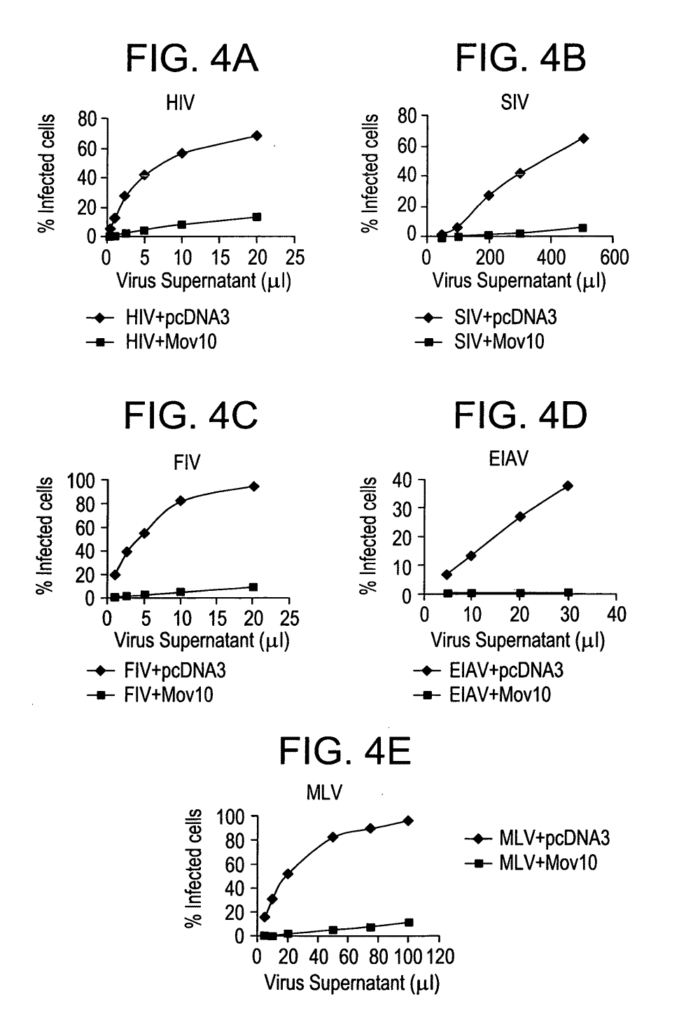

Whether Mov10 could also inhibit the generation of replication-competent HIV-1 from primary human CD4⁺ T cells was investigated. For this experiment, highly purified CD4⁺ T cells were nucleofected with a replication-competent, CCR5-tropic HIV-1 plasmid (R5.HIV.GFP) in the presence of a Mov10 expression plasmid or control plasmid (pcDNA3). The virus produced from CD4⁺ T cells from this transfection was in turn used to infect CCR5⁺ Hut78 T cells (experimental setup shown in FIG. 3A). Virus production in primary cells was slightly impaired in the presence of Mov10 (FIG. 3B). However, when supernatants containing identical levels of p24 were applied to Hut78 cells, a significant reduction in HIV-1 infectivity due to Mov10 overexpression was observed (FIG. 3C). Next, viral plasmids for other retroviruses, SIV, MLV, FIV and EIAV, along with the Mov10 expression plasmid or empty vector to produce the respective viruses in presence or absence of Mov10 were co-transfected. Their infectivity on HeLa cells was tested and found that in the presence of Mov10, similar to HIV-1, infectivity of all of the tested lentiviruses and the retrovirus were profoundly suppressed (FIG. 4).

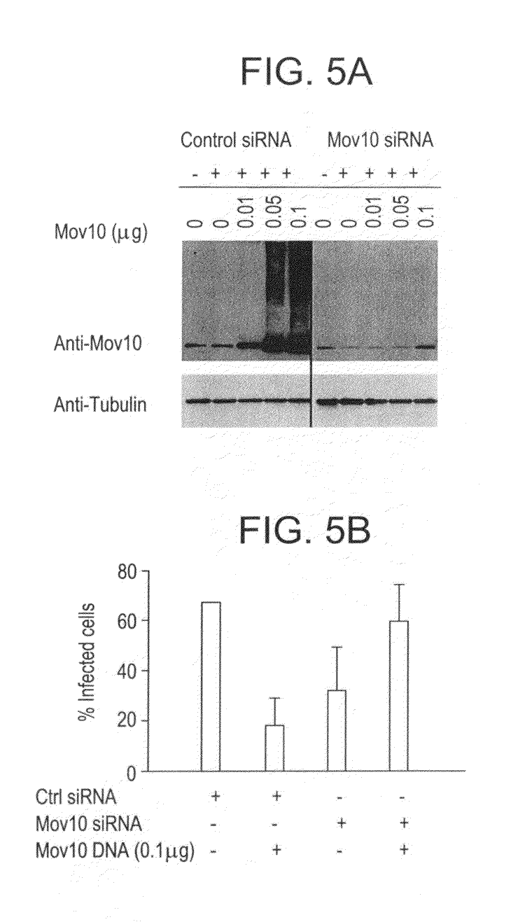

Because overexpression of Mov10 impaired HIV-1 infectivity, whether endogenously expressed Mov10 was also inhibitory to HIV-1 replication. To address this Mov10 expression was silenced in producer cells using siRNAs targeting Mov10. Reduced levels of Mov10 also suppressed HIV-infectivity (FIG. 5), indicating a positive role for Mov10 in the generation of optimally infectious virions. To further test whether Mov10 levels were indeed critical or whether the siRNA was acting “off target,” Mov10 was ectopically expressed in siRNA-treated cells from which HIV-1 was produced. Because high levels of Mov10 would be inhibitory to HIV-1, the knockdown cells were complemented with a wild-type form of Mov10 that remained susceptible to the siRNA pool to restrict increased expression. In the absence of Mov10 siRNA, transfection of increasing amounts of the Mov10 expression plasmid leads to a dramatic increase of Mov10 levels even at relatively low input DNA amounts (FIG. 5A, left). At the highest levels of Mov10 (0.1 μg DNA), the specific infectivity of HIV-1 is diminished (FIG. 5B). In the presence of Mov10 siRNA, Mov10 levels, relative to protein loading controls, are less dramatically increased by Mov10 plasmid transfection (FIG. 5A, right). Without Mov10 plasmid transfection, siRNA targeting of endogenous Mov10 led to a 2-fold reduction in HIV-1 particle infectivity (FIG. 5B). The restoration of Mov10 expression to endogenous levels (0.1 μg DNA) was sufficient to increase HIV-1 specific infectivity. These data indicate that endogenous Mov10 aids in HIV-1 replication and that slight variation from the wild type level of Mov10 can drastically affect the infectivity of HIV-1.

Mov10 Decreases Infectivity of HIV-1

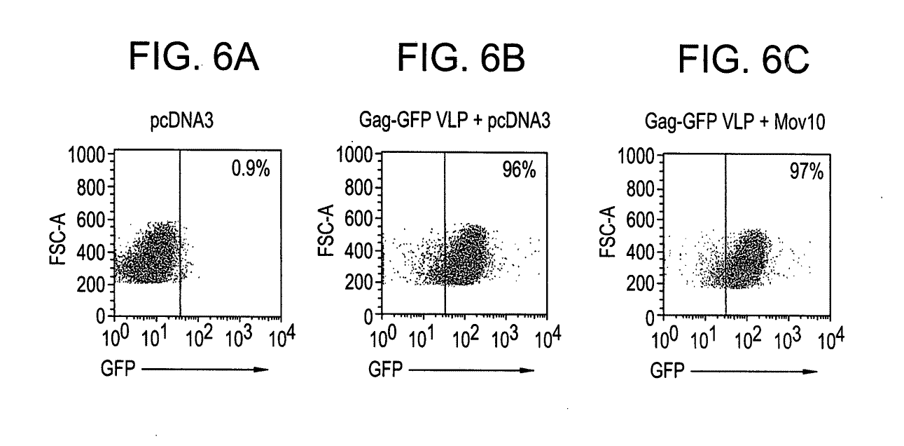

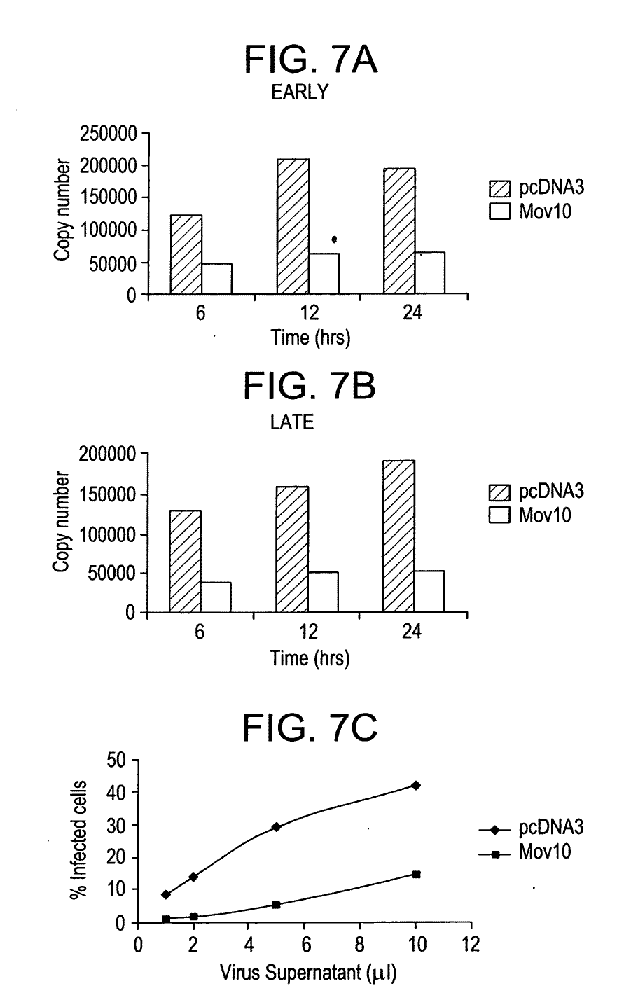

In order to determine how Mov10 reduces the infectivity of HIV-1, the early events in the lifecycle of HIV-1 produced in the presence of perturbed Mov10 levels were analyzed. Whether Mov10 interfered with the incorporation of viral glycoproteins in HIV-1 particles was determined. Virion-like particles (VLP) that express GFP through fusion to Gag and trans-incorporate VSV-G were constructed. The VLPs were then used to assess their binding capacity to a T cell line by analysis for GFP. When the target cells were analyzed by flow cytometry, there was no decrease in the ability of VLPs produced in the presence of MovlO to bind to Jurkat cells (FIG. 6). Whether Mov10-treated HIV-1 has defects during its early, post-entry stages was evaluated. Quantitative real-time PCR was used to analyze the early and late HIV-1 reverse transcripts (FIG. 7). Compared to virus from cells expressing a control plasmid, virus from Mov10-overexpressing cells was 80% less efficient in synthesis of early reverse transcripts (minus-strand strong-stop DNA). This indicates that the defect induced by Mov10 may manifest prior to the initiation of reverse transcription during the early stages of post-entry replication of the virus.

Mov10's Anti-HIV-1 Activity is Independent of its Putative RNA Helicase Domain.

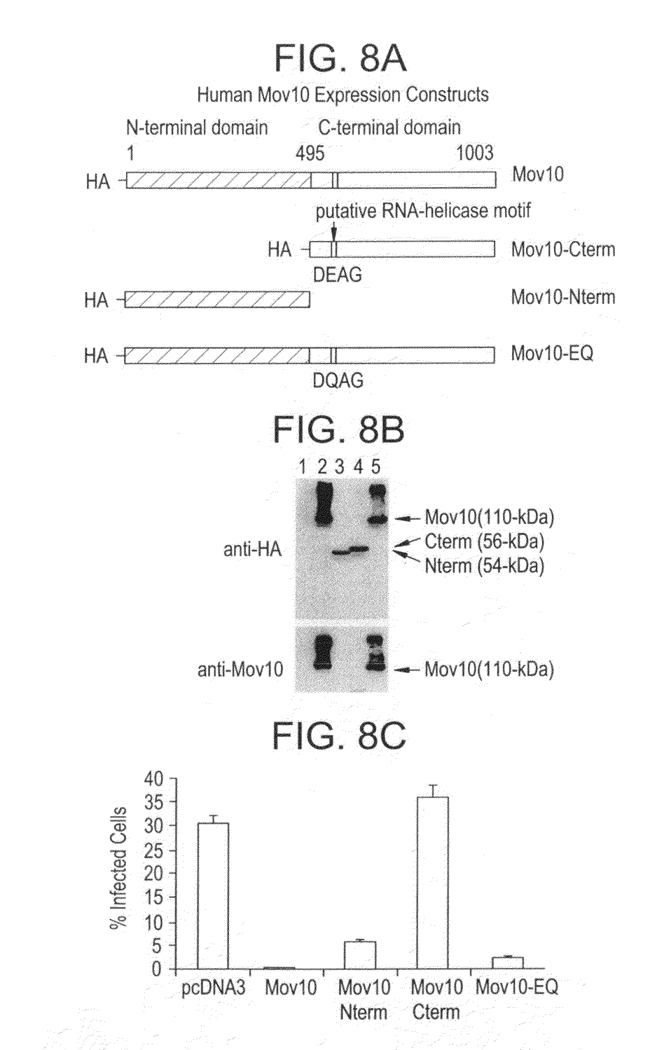

Because a potential RNA helicase domain has been identified for Mov10 and because RNA helicases have been implicated as modulators of HIV-1 replication (Ma et al., (2008) Virology 375: 253-264; Zhou et al., (2008) Virology 372: 97-106), Mov10's antiviral activity may be due to the presence of its putative helicase domain. To analyze the roles of the particular domains of Mov10, the protein into N-terminal and C-terminal portions (FIG. 8A) were split. The C-terminal half of the protein contains the putative RNA helicase domain of the protein while the N-terminal half is not yet characterized (Dalmay et al., (2001) Embo J 20: 2069-2078; Cordin et al., (2006) Gene 367: 17-37; de la Cruz et al., (1999) Trends Biochem Sci 24: 192-198). In addition a point mutation of a motif predicted to be essential for ATP binding to the potential helicase (FIG. 8A) was generated, based upon previous reports of an inactivating mutation in an RNA helicase (Askjaer et al., (2000) J Biol Chem 275: 11561-11568). HIV-1 particles were produced by co-transfection of 293T cells in the presence of either empty vector, Mov10, Mov10 N-terminus, Mov10 C-terminus or the helicase domain mutant of Mov10. All HA-tagged proteins were detectably expressed in cells (FIG. 8B). Examining the infectivity of the produced virions demonstrated that both the N-terminal half of Mov10, which lacks the helicase domain, and the helicase domain point mutant diminished infectivity of HIV-1 particles nearly as well as wild type Mov10 (FIG. 8C). By contrast, the C-terminal domain alone had no effect. These data show that the N-terminal portion of the protein was required for Mov10-mediated virus suppression and that the putative RNA helicase domain did not contribute to Mov10's antiviral activity under these experimental conditions.

These data demonstrate that the RNA helicase Mov10 may modulate the production of infectious HIV-1. Ectopic expression of Mov10 diminishes per-particle infectivity resulting in virus impaired at an early step of infection in target cells. This effect is observed in both primary and transformed cells using HIV-1 single-cycle vectors or replication-competent genomes. Importantly, endogenous Mov10 contributes to HIV-1 replication as virions produced from cells that have been depleted of Mov10 are also less infectious. These data demonstrate that Mov10 is at a critical nexus of HIV-1 replication and perturbation of this factor is restrictive to the virus. Notably, other retroviruses are also sensitive to elevated expression of Mov10.

Mov10 has been found in association with Ago1 and Ago2 in the RISC, together with TNRC6B, which are also found to localize to P-bodies (Chendrimada et al., (2007) Nature 447: 823-828; Baillat et al., Mol Cell Biol 29: 4144-4155). In addition, ectopically expressed Mov10 appears to be enriched in P-bodies (Meister et al., (2005) Curr Biol 15: 2149-2155), similar to APOBEC3G (Niewiadomska et al., (2007) J Virol 81: 9577-9583). P-body components are essential for retrotransposition of yeast Ty1 and Ty3 elements. (Checkley et al., Mol Cell Biol 30: 382-398; Beliakova-Bethell et al., (2006) Rna 12: 94-101) They may also play a role in retroviral replication. Perturbation of P-bodies may affect cellular RNA metabolism and thus limit HIV-1 production. Unlike GW 182 or other P-body components, Mov10 is not known to be essential to the genesis or turnover of P-bodies. These data demonstrate that the role of Mov10 in HIV-1 replication may be independent of RNA metabolism.

The incorporation of APOBEC3G by different retroviruses indicates that they may traffic via P-bodies during viral production. Indeed, Mov10 was also reported to be incorporated in HIV-1 particles. (Ott, (2008) Rev Med Virol 18: 159-175) The presence of a RISC component in HIV-1 particles is provocative given that RISC may be physically associated with multivesicular bodies (Gibbings et al., (2009) Nat Cell Biol 11: 1143-1149) that are essential for the production and release of retroviruses. Retroviral RNA modifications or association with the viral core components may require RISC or P-body machinery, which in turn may be disrupted by modulating Mov10 expression levels.

Silencing a portion of Mov10 expression in producer cells also reduced infectivity of HIV-1, albeit with much lower potency compared to inhibition seen in over-expression experiments. It may be that removal of Mov10 from virions or the absence of functional Mov10 in virus-producers cells underlies the loss in infectivity. Alternatively, perturbation of Mov10 levels may disrupt other components of the P-body machinery, that are required for viral RNA processing and assembly. It will also be important to determine the physiological function of Mov10 in primary human T cells and macrophages, which are the natural targets of HIV-1.

Mov10 is an attractive therapeutic target given the small window in which cellular levels are compatible with production of infectious HIV-1. Elevated levels of Mov10 in primary cells are sufficient to hinder HIV-1 replication, and it is conceivable that pharmacological treatment of these cells with drugs that stabilize or increase P-body numbers may in turn increase Mov10 levels. Notably, Mov10 lacking an RNA interaction domain through limited mutagenesis or complete removal of the C-terminal domain is antiviral when ectopically expressed. Thus fragments of the protein may be used as antivirals and potentially minimize effects on the cell biology. The N-terminal domain also provides an attractive tool with which to select for HIV-1 resistance to better understand the interaction of Mov10 with the virus. The N-terminal domain may be interacting with and disrupting other components of the P-body machinery, or it may have a yet to be identified function in RNA processing.

The effect of Mov10 depletion or overexpression on HIV-1 replication is reminiscent of the effect that the ESCRT component Tsg101 can have on HIV-1 production (Goila-Gaur et al., (2003) J Virol 77: 6507-6519). Depletion of Tsg101 impairs HIV-1 release, overexpression of Tsg101 interferes with HIV-1 release, and expression of Tsg101 fragments prevents HIV-1 Gag interactions with Tsg101 or other ESCRT components thus blocking virus release. More proteins will likely be discovered that exhibit similar characteristics to Mov10—these being a narrow expression window in which HIV-1 is capable of reproducing. HIV-1's requirement of an optimal level of Mov10 during virion production provides excellent, potential targets for therapy because perturbation of their levels in either direction reduces HIV-1's ability to reproduce.

P-Body Proteins such as Mov10 Inhibit Retrotransposition

Nearly 50% of the human genome is composed of fossils from the remains of past transposable element duplication. Mobilization continues in the genomes of humans but is now restricted to retrotransposons, a class of mobile elements that move via a copy and paste mechanism. Currently active retrotransposable elements include Long INterspersed Elements (LINEs), Short INterspersed Elements (SINEs) and SVA (SINE/VNTR/Alu) elements. Retrotransposons are responsible for creating genetic variation and on occasion, disease-causing mutations, within the human genome. Approximately 0.27% of all human disease mutations are attributable to retrotransposable elements. Different mechanisms of genome alteration created by retrotransposable elements include insertional mutagenesis, recombination, retrotransposition-mediated and gene conversion-mediated deletion, and transduction. There are many mutational mechanisms for retrotransposable elements. However, their contribution to genetic variation within humans is still being resolved. (See, Callinan et al., “Retrotransposable Elements and Human Disease,” Volff (ed): Genome and Disease. Genome Dyn. Basel, Karger, 2006, vol. 1, pp. 104-115)

Genetic instability is one of the principal hallmarks and causative factors in cancer. Human transposable elements (TE) have been reported to cause several types of cancer through insertional mutagenesis of genes critical for preventing or driving malignant transformation. In addition to retrotransposition-associated mutagenesis, TEs have been found to contribute even more genomic rearrangements through non-allelic homologous recombination. TEs also may generate a wide range of mutations difficult to trace to mobile elements, including double strand breaks that may trigger mutagenic genomic rearrangements. Genome-wide hypomethylation of TE promoters and significantly elevated TE expression in almost all human cancers often accompanied by the loss of DNA sensing and repair pathways indicate the negative impact of mobile elements on genome stability. The biological consequences of elevated retroelement expression, such as the rate of their amplification, in human cancers has been demonstrated. (See, Belancio et al., Seminars in Cancer Biology 20 (2010) 200-210)

Blocking retroelements may be critical to prevent aberrant retroelement insertions into the genome. In fact it is estimated that, a single retrolement (Alu elements) is responsible for 0.1% of all human diseases with genetic causes. (Deininger et al., (1999) Molecular Genetics and Metabolism 67: 183-93) These uncontrolled transposon insertions have also been implicated in various forms of cancer. Enhancing the activity of a p-body protein such as Mov10 or associated proteins may inhibit or suppress unwanted jumping and insertion of retroelements.

### Administration of Therapeutic Compositions

According to the present invention, the component or components of a therapeutic composition of the invention may be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration.

In some instances, the components or composition are administered to prevent or treat AIDS and are introduced by injection into the blood. In another embodiment, the therapeutic components or composition can be delivered in a vesicle, in particular a liposome (See, Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

In yet another embodiment, the therapeutic compound can be delivered in a controlled release system. For example, an antibody as described above, may be administered using intravenous infusion, an implantable osmotic pump, a transdermal patch, liposomes, or other modes of administration. In one embodiment, a pump may be used (See, Langer, supra; Sefton, CRC Crit. Ref Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of a therapeutic target, e.g., the brain, thus requiring only a fraction of the systemic dose (See, e.g., Goodson, in Medical Applications of Controlled Release, supra, vol. 2, pp. 115-138 (1984)). Other controlled release systems are discussed in the review by Langer, Science 249:1527-1533 (1990).

Thus, a therapeutic composition of the present invention can be delivered by intravenous, intraarterial, intraperitoneal, intramuscular, or subcutaneous routes of administration. Alternatively, the therapeutic composition , properly formulated, can be administered by nasal or oral administration. A constant supply of the therapeutic composition can be ensured by providing a therapeutically effective dose (i.e., a dose effective to induce metabolic changes in a subject) at the necessary intervals, e.g., daily, every 12 hours, etc. These parameters will depend on the severity of the disease or condition being treated, other actions, such as diet modification, that are implemented, the weight, age, and sex of the subject, and other criteria, which can be readily determined according to standard good medical practice by those of skill in the art. A subject in whom administration of the therapeutic composition is an effective therapeutic regiment for AIDS is preferably a human, but can be a primate with a related viral condition. Thus, as can be readily appreciated by one of ordinary skill in the art, the methods and pharmaceutical compositions of the present invention are particularly suited to administration to a number of animal subjects including humans.

Where administration of an antagonist to Mov10 is administered to prevent or treat AIDS, it is preferred for it to be introduced by injection into the blood. The antagonist may be a specific antibody raised against Mov10 or a mimic to Mov10 that competitively competes with Mov10.

In another embodiment, the therapeutic compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.) To reduce its systemic side effects, this may be a preferred method for introducing an antagonist to Mov10.

In yet another embodiment, the therapeutic compound can be delivered in a controlled release system. For example, an antibody as described above, may be administered using intravenous infusion, an implantable osmotic pump, a transdermal patch, liposomes, or other modes of administration. In one embodiment, a pump may be used (see Langer, supra; Sefton, CRC Crit. Ref Biomed. Eng. 14:201 (1987); Buchwald et al., Surgery 88:507 (1980); Saudek et al., N. Engl. J. Med. 321:574 (1989)). In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of a therapeutic target, e.g., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer [Science 249:1527-1533 (1990)].

Thus, a therapeutic composition of the present invention can be delivered by intravenous, intraarterial, intraperitoneal, intramuscular, or subcutaneous routes of administration. Alternatively, the therapeutic composition , properly formulated, can be administered by nasal or oral administration. A constant supply of the therapeutic composition can be ensured by providing a therapeutically effective dose (i.e., a dose effective to induce metabolic changes in a subject) at the necessary intervals, e.g., daily, every 12 hours, etc. These parameters will depend on the severity of the disease or condition being treated, other actions, such as diet modification, that are implemented, the weight, age, and sex of the subject, and other criteria, which can be readily determined according to standard good medical practice by those of skill in the art.

A subject in whom administration of the therapeutic composition is an effective therapeutic regiment for AIDS is preferably a human, but can be a primate with a related viral condition. Agents that cause an increase in Mov10 can be used in therapeutic compositions. Thus, as can be readily appreciated by one of ordinary skill in the art, the methods and pharmaceutical compositions of the present invention are particularly suited to administration to a number of animal subjects including humans.

### Transgenic Vectors and Effecting Expression

In one embodiment, a gene encoding a therapeutic compound can be introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus macrophage can be specifically targeted. Examples of particular vectors include, but are not limited to, an attenuated adenovirus vector, such as the vector described by Stratford-Perricaudet et al. J. Clin. Invest. 90:626-630 (1992)), and a defective adeno-associated virus vector (Samulski et al., J. Virol. 61:3096-3101 (1987); Samulski et al., J. Virol. 63:3822-3828 (1989)).

In another embodiment the gene or antigene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120; Temin et al., U.S. Pat. No. 5,124,263; International Patent Publication No. WO 95/07358, published Mar. 16, 1995, by Dougherty et al.; and Kuo et al., 1993, Blood 82:845. Targeted gene delivery is described in International Patent Publication WO 95/28494, published October 1995.

Alternatively, the vector can be introduced in vivo by lipofection (Feigner, et. al., Proc. Natl. Acad. Sci. U.S.A. 84:7413-7417 (1987); see Mackey, et al., Proc. Natl. Acad. Sci. U.S.A. 85:8027-8031 (1988); Feigner and Ringold, Science 337:387-388 (1989)). Lipids may be chemically coupled to other molecules for the purpose of targeting (See, Mackey, et. al., supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (See, e.g., Wu et al., J. Biol. Chem. 267:963-967 (1992); Wu and Wu, J. Biol. Chem. 263:14621-14624 (1988); Hartmut et al., Canadian Patent Application No. 2,012,311, filed Mar. 15, 1990).

### Antibodies to Mov10

Mov10 or a fragment or homolog thereof may be used as an immunogen to generate antibodies that recognize the Mov10 or a fragment or homolog thereof. Such antibodies include but are not limited to polyclonal, monoclonal, chimeric, single chain, Fab fragments, and an Fab expression library. The Mov10 or a fragment or homolog thereof of the invention may be cross reactive, e.g., they may recognize Mov10 from different species. Polyclonal antibodies may have greater likelihood of cross reactivity. Alternatively, an antibody of the invention may be specific for a single form of Mov10. Preferably, such an antibody is specific for human Mov10.

Various procedures known in the art may be used for the production of polyclonal antibodies to Mov10 or a fragment, derivative or analog thereof. For the production of antibody, various host animals can be immunized by injection with the Mov10 agent, or a derivative (e.g., fragment or fusion protein) thereof, including but not limited to rabbits, mice, rats, sheep, goats, etc. In one embodiment, the Mov10 agent or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the Mov10 or a fragment, analog, or derivative thereof, any technique that provides for the production of antibody molecules by continuous cell lines in culture may be used. These include but are not limited to the hybridoma technique originally developed by Kohler and Milstein (Nature 256:495-497 (1975)), as well as the trioma technique, the human B-cell hybridoma technique (Kozbor et al., Immunology Today 4:72 1983); Cote et al., Proc. Nati. Acad. Sci. U.S.A. 80:2026-2030 (1983)], and the EBV-hybridoma technique to produce human monoclonal antibodies (Cole et al., in Monoclonal Antibodies and Cancer Therapy, Alan R. Liss, Inc., pp. 77-96 (1985)). In an additional embodiment of the invention, monoclonal antibodies can be produced in germ-free animals utilizing recent technology described in PCT/US90/02545. In fact, according to the invention, techniques developed for the production of “chimeric antibodies” (Morrison et al., J. Bacteriol. 159:870 (1984); Neuberger et al., Nature 312:604-608 (1984); Takeda et al., Nature 314:452-454 (1985)) by splicing the genes from a mouse antibody molecule specific for a Mov10 agent together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (U.S. Pat. Nos. 5,476,786 and 5,132,405 to Huston; U.S. Pat. No. 4,946,778) can be adapted to produce MOV10-specific single chain antibodies. An additional embodiment of the invention utilizes the techniques described for the construction of Fab expression libraries (Huse et al., Science 246:1275-1281 (1989)) to allow rapid and easy identification of monoclonal Fab fragments with the desired specificity for a Mov10 protein, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

### Methods for Screening Drug Libraries

### Identification of Potentially Therapeutic Compounds

Identification and isolation of a gene encoding a Mov10 of the invention provides for expression of Mov10 in quantities greater than can be isolated from natural sources, or in indicator cells that are specially engineered to indicate the activity of Mov10 protein expressed after transfection or transformation of the cells. Accordingly, the present invention contemplates a method for identifying agonists and antagonists of Mov10 using various screening assays known in the art. In one embodiment, such agonists or antagonists competitively inhibit Mov10,

Any screening technique known in the art can be used to screen for antagonists of Mov10. The present invention contemplates screens for small molecule ligands or ligand analogs and mimics, as well as screens for natural ligands that bind to and antagonize such activity in vivo. For example, natural products libraries can be screened using assays of the invention for molecules that antagonize Mov10. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Another approach uses recombinant bacteriophage to produce large libraries. Using the “phage method” (Scott and Smith, 1990, Science 249:386-390 (1990); Cwirla, et al., Proc. Natl. Acad. Sci., 87:6378-6382 (1990); Devlin et al., Science, 249:404-406 (1990)), very large libraries can be constructed. A second approach uses primarily chemical methods, of which the Geysen method (Geysen et al., Molecular Immunology 23:709-715 (1986); Geysen et al. J. Immunologic Method 102:259-274 (1987)) and the method of Fodor et al. Science 251:767-773 (1991)) are examples. Furka et al. 14 th International Congress of Biochemistry, Volume 5, Abstract FR:013 (1988); Furka, Int. J. Peptide Protein Res. 37:487-493 (1991)), Houghton (U.S. Pat. No. 4,631,211, issued December 1986) and Rutter et al. U.S. Pat. No. 5,010,175, issued Apr. 23, 1991 describe methods to produce a mixture of peptides that can be tested as agonists or antagonists.

In another aspect, synthetic libraries (Needels et al., Proc. Natl. Acad. Sci. USA 90:10700-4 (1993); Ohlmeyer et al., Proc. Natl. Acad. Sci. USA 90:10922-10926 (1993); Lam et al., International Patent Publication No. WO 92/00252; Kocis et al., International Patent Publication No. WO 9428028, each of which is incorporated herein by reference in its entirety), and the like can be used to screen for Mov10 ligands according to the present invention.

The screening can be performed with recombinant cells that express the Mov10, or alternatively, using purified protein, e.g., produced recombinantly. For example, the ability of a labeled, soluble or solubilized that includes the ligand-binding portion of the molecule, to bind ligand can be used to screen libraries, as described in the references cited above. In addition, orphan chemokines, potential chemokines, or potential ligands that are obtained from random phage libraries or chemical libraries, as described herein, can be tested by any of the numerous assays well known in the art and exemplified herein. In one particular embodiment of the present invention, an in situ assay is employed in which the detection of the calcium signaling elicited by the binding of a potential chemokine to a chemokine receptor is indicative of the chemokine having specificity for the chemokine receptor, and therefore is a ligand.

### Transgenic Vectors and Inhibition of Expression

In one embodiment, a gene encoding a Mov10, or antisense or ribozyme specific for Mov10 mRNA (termed herein an “antigene”) or a reporter gene can be introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus macrophage can be specifically targeted. Examples of particular vectors include, but are not limited to, a defective herpes virus 1 (HSV1) vector [Kaplitt et al., Molec. Cell. Neurosci. 2:320-330 (1991)], an attenuated adenovirus vector, such as the vector described by Stratford-Perricaudet et al. J. Clin. Invest. 90:626-630 (1992), and a defective adeno-associated virus vector (Samulski et al., J. Virol. 61:3096-3101 (1987); Samulski et al., J. Virol. 63:3822-3828 (1989)).

In another embodiment the gene or antigene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120; Temin et al., U.S. Pat. No. 5,124,263; International Patent Publication No. WO 95/07358, published Mar. 16, 1995, by Dougherty et al.; and Kuo et al., 1993, Blood 82:845. Targeted gene delivery is described in International Patent Publication WO 95/28494, published October 1995.

Alternatively, the vector can be introduced in vivo by lipofection (Feigner, et. al., Proc. Natl. Acad. Sci. U.S.A. 84:7413-7417 (1987); see Mackey, et al., Proc. Natl. Acad. Sci. U.S.A. 85:8027-8031 (1988); Feigner and Ringold, Science 337:387-388 (1989)]. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., J. Biol. Chem. 267:963-967 (1992); Wu and Wu, J. Biol. Chem. 263:14621-14624 (1988); Hartmut et al., Canadian Patent Application No. 2,012,311, filed Mar. 15, 1990).

As noted above, the present invention extends to the preparation of antisense nucleotides and ribozymes that may be used to interfere with the expression of Mov10 at the translational level. This approach utilizes antisense nucleic acid and ribozymes to block translation of a specific mRNA, either by masking that mRNA with an antisense nucleic acid or cleaving it with a ribozyme. Such antisense or ribozyme nucleic acids may be produced chemically, or may be expressed from an “antigene.”

Antisense nucleic acids are DNA or RNA molecules that are complementary to at least a portion of a specific mRNA molecule (see Marcus-Sekura, Anal. Biochem. 172:298 (1988)). In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into organ cells. Antisense methods have been used to inhibit the expression of many genes in vitro (Marcus-Sekura, Anal. Biochem. 172:298 (1988); Hambor et al., J. Exp. Med. 168:1237 (1988)). Preferably synthetic antisense nucleotides contain phosphoester analogs, such as phosphorothiolates, or thioesters, rather than natural phosphoester bonds. Such phosphoester bond analogs are more resistant to degradation, increasing the stability, and therefore the efficacy, of the antisense nucleic acids.

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, J. Am. Med. Assoc. 260:3030 (1988)). Because they are sequence-specific, only mRNAs with particular sequences are inactivated.

The DNA sequences encoding the Mov10 can be used to prepare antisense molecules against and ribozymes that cleave mRNAs for Mov10, thus inhibiting expression of the gene encoding the Mov10, which can reduce the level of HIV translocation in macrophages and T cells.

### DETAILED DESCRIPTION OF THE PREFERRED EMBODIMENTS

### EXAMPLE 1

### Materials and Methods

Cell Purifications and Culture. Blood samples were obtained from anonymous healthy donors as buffy coats (New York Blood Center). New York Blood Center obtains written informed consent from all participants involved in the study. Because all the samples were sent as anonymous, the Institutional Review Board at New York University medical center determined that the study was exempt from further ethics approval requirement. Peripheral Blood Mononuclear cells (PBMC) were isolated with Ficoll-Hypaque (Amersham Pharmacia). CD4⁺ T cells were isolated from PBMC using magnetic bead sorting (Invitrogen, Dynabeads). Purified CD4⁺ T cells were activated using anti-CD3/CD28 coated beads (Dynabeads, Invitrogen) and cultured in RPMI media (Life Technologies) with 10% fetal calf serum (FCS; Atlanta Biologicals) and supplemented with IL-2 (200 U/ml). Jurkat and Hut78 cells were also grown in RPMI-10% FCS media. HEK293T and HeLa cell lines were maintained in DMEM supplemented with 10% FCS, 100 U/ml penicillin and 0.1 mg/ml streptomycin.

Plasmids and Mov10 mutants. The HIV-RFP plasmid was constructed from the HIV-EGFP plasmid (Unutmaz et al., (1999) J Exp Med 189: 1735-1746) and has been described previously (Lee et al., in press). HIV Vif gene cloned in pcDNA 3 (Nguyen et al., (2004) Virology 319: 163-175) was obtained from NIH AIDS reagent repository. Wild type Mov10 (NCBI accession number BC009312.2, cDNA obtained from Origene) and mutant Mov10s, Mov10-Nterm (corresponds to amino acids 1-495), Mov10-Cterm (corresponds to amino acids 496-1003) and Mov10.EQ (putative helicase motif mutant) were PCR amplified and subcloned into the pcDNA3 vector that, contained an in frame 5′ HA tag, using BamHI and NotI restriction sites to create HA-tagged mutants. Following primers were used for PCR amplifications: For full-length Mov10: 5′ TAC GCC GGA TCC CCC AGT AAG TTC AGC TGC CGG CAG-3′ (SEQ ID NO: 2) CGT TAG GCG GCC GCT CAG AGC TCA TTC CTC CAC TCT GGC TCC. (SEQ ID NO: 3) For Nterm mutant: 5′ TAC GCC GGA TCC CCC AGT AAG TTC AGC TGC CGG CAG-3′ (SEQ ID NO: 4) CGT TAG GCG GCC GCT CAC CGG TCG TAC AGC TTG AGT TTC ACA (SEQ ID NO: 5). For Cterm Mov10: 5′TAC GCC GGA TCC AGT CTG GAG TCA AAC CCA GAG CAG, 3′ (SEQ ID NO: 6) CGT TAG GCG GCC GCT CAG AGC TCA TTC CTC CAC TCT GGC TCC (SEQ ID NO: 7). The point mutation in the DEAG sequence of Mov10 was created by replacing glutamate residue at position 646 with glutamine (DEAG to DQAG) and named Mov10-EQ. APOBEC3G was similarly subcloned into pcDNA3 vector for transfections. The pNL4.3.R-.E-.Luc vector was provided by Dr. Nathaniel Landau (New York University). Replication competent CCR5-tropic HIV (R5.HIV) expressing GFP was previously described (Oswald-Richter et al., (2007) PLoS Pathog 3: e58). Virion-like particles (VLPs) were generated using plasmid expressing HIV-1 Gag fused to GFP (kind gift of Dr. Paul Spearman, Emory University).

Virus Stocks. Virus stocks were produced by DNA transfection on monolayer cultures of 293T cells grown in six-well plates (Corning) using either Hilymax (Dojindo) or Lipofectamine 2000 (Invitrogen) transfection reagents. To produce HIV-1 vectors (VSVG.HIV-RFP or VSVG.NL4.3.R-.E-.Luc), each well of 293T cells in a six-well plate was cotransfected with 3 μg of HIV and 0.5 μg of p-L-VSV-G (Bartz et al., (1997) Methods 12: 337-342). In experiments where Mov10 was also cotransfected, the plasmid was used in amounts ranging from 0.5 μg to 0.002 μg, which corresponds to ratios of HIV-1/Mov10 of 1/6 to 1/1500. 293T cells were cotransfected with 2.5 μg of pMIGR1 (Pear et al., (1998) Blood 92: 3780-3792), 1.5 μg of pJK3 (31), 0.5 μg of pCMV-Tat and 1 μg of p-L-VSV-G plasmids (Bartz et al., (1997) Methods 12: 337-342) to produce the murine leukemia virus (MLV) stock; with 2 μg of pV1EGFP (SIV vector) and 2 μg of pUpSVOΔψ (SIV structural proteins) and 0.5 μg of pCMV-VSVG to produce the simian immunodeficiency virus (SIV) stock; with 2 μg of pGinSin (FIV vector) and 2 μg of pFP93 (FIV structural proteins) and 0.5 μg CMV-VSVG to produce the feline immunodeficiency virus (FIV) stock; and with 2 μg of p6.1G3CeGFPW (EIAV vector), 2 μg of pEV53B (EIAV structural proteins) (kindly provided by John Olsen) and 0.5 μg of pCMV-VSV-G to produce the equine infectious anemia virus (EIAV) stocks. To examine the effects of Mov10 on viruses produced, the 293T cells were also cotransfected with either the empty pcDNA3 plasmid as control or the pcDNA3-HA-Mov10 plasmid. Culture supernatants from the 293T cells were collected 48 h post-transfection, clarified by low-speed centrifugation (1,000×g, 10 min), and filtered through 0.45 μm pore-size sterile filters.

HIV-1 p24 ELISA. For the HIV-1 vectors, the clarified supernatants were analyzed for p24 antigen concentration by enzyme-linked immunosorbent assay (PerkinElmer) following manufacturer's instructions. HRP levels were detected via colorimetry and quantified following manufacturer's protocol on an Envision 96-well plate reader (PerkinElmer). HIV-1 capsid monoclonal antibody was obtained through the NIH AIDS Research and Reference Reagent Program, Division of AIDS, NIAID, NIH (183-H12-5C, contributed by Bruce Chesebro and Hardy Chen). Secondary antibodies included HRP-conjugated goat anti-mouse, goat anti-human, and goat anti-rabbit antibodies (GE Healthcare).

Infection Assays. Infectivities of non-HIV retroviruses were determined either by titration of virus supernatants on HeLa cells and of HIV-1 on Jurkat or Hut78 cells using virus samples normalized by p24 (capsid) levels. The expression of GFP or RFP following infection by the HIV-1, MLV, SIV, FIV and EIAV viruses was measured by fluorescence-activated cell sorter (FACS) analysis (FACSCalibur, Becton Dickinson). The percent infected cells represents the percentage of GFP-positive or RFP-positive cells in the cell population. Alternatively, for luciferase reporter viruses, Jurkat cells (1.5×10⁴ per well) were infected for 3 days with VSV-G-pseudotyped NL4.3.R-.E-.Luc (VSVG.HIV.Luc) and luciferase activity was measured by LucLite kit from PerkinElmer according to the manufacturer's protocol using an Envision 96-well plate reader (PerkinElmer).

Knockdown of Endogenous Mov10. Transient silencing of target genes was achieved by transfecting the gene-specific siRNAs (Dharmacon, ON-TARGETplus SMARTpool L-014162-00) into 293T cells using Oligofectamine (Invitrogen). 50 nM of the nontargeting control siRNA or gene-specific siRNA was transfected into 293T cells 48 h prior to plasmid transfections. Knockdown of Mov10 protein was confirmed by Western blot.

Nucleofection of Primary T Cells. One day after activation, sorted human CD4⁺ cells were nucleofected using the Amaxa Nucleofector System (Lonza) with R5.HIV.GFP plasmid (1 μg) and either Mov10 or pcDNA3 plasmid (0.5 μg). 48 hours after nucleofection, viral supernatants were collected and analyzed for p24 concentration by ELISA as previously described (Oswald-Richter et al., (2007) PLoS Pathog 3: e58). Aliquots containing 100 pg of p24 were then used to infect 1.5×10⁴ CCR5⁺ Hut78 cells. After 3 days of culture percent of infected cells was analyzed by FACS.

Real-Time PCR Analysis of Virus Infection. The 293T-derived HIV-1 virions were treated with 50 U/ml DNaseI (Roche) for 30 minutes. These DNaseI treated HIV-1 virions were standardized by p24 concentration and then used to infect HeLa cells for different times. At each time points cells were collected, lysed and total DNA was extracted using the QIAmp Blood Mini Kit (Qiagen). As a control for plasmid contamination, an equivalent amount of virus was either boiled for 10 minutes before infecting cells or the reverse transcriptase inhibitor efavirenz (100 nM) was added at the time of infection. Samples were assayed by Real-Time PCR using Platinum qPCR SuperMix-UDG (Invitrogen) with primers, probes and PCR conditions as described previously (Julias et al., (2001) J Virol 75: 6537-6546). Duplicate samples of serial dilutions of plasmid DNAs containing the target sequences were used to generate a standard curve, which was used for quantification of PCR products.

Western Blot Analysis. Transfected cells were lysed and solubilized in RIPA buffer (Sigma) (50 mM Tris-HCl, pH 8.0, with 150 mM sodium chloride, 1.0% Igepal CA-630 (NP-40), 0.5% sodium deoxycholate, and 0.1% sodium dodecyl sulfate). The cell lysates were then mixed with a 2× Laemmli sample loading buffer (BioRad) (62.5 mM Tris-HCl, pH 6.8, 25% glycerol, 2% SDS, 0.01% bromophenol blue and 5% 2-mercaptoethanol), boiled and then samples were loaded and separated on 10.0% polyacrylamide gels containing SDS. Following electrophoresis, proteins were transferred to a PVDF membrane by electroblotting and incubated for 1 hr at room temperature in blocking buffer (5% nonfat dry milk in PBS). The blocked blot was exposed to the appropriate primary antibody in blocking buffer with constant mixing. After extensive washing, bound antibodies were detected by chemiluminescence using horseradish peroxidase-conjugated, species-specific, secondary antibodies as described by the manufacturer (GE Healthcare). The following antibodies were used for the western blot analysis: anti-Mov10 (Proteintech Group, Inc.), anti-HA epitope (Sigma) and anti-α-tubulin (Sigma), using the manufacturer's recommended antibody concentrations.

### Results

Overexpression of Mov10 reduces the specific infectivity of HIV-1. In order to identify proteins that interact with APOBEC3G, a mass spectrometry analysis of proteins co-immunoprecipitated with APOBEC3G from primary CD4⁺ T cells or 293T cells was performed. It was revealed that the putative RNA helicase, Mov10, was reproducibly and predominantly associated with high-molecular weight APOBEC3G preparations. This finding was in line with observations from other reports that detected Mov10 in similar mass spectrometry analysis (Gallois-Montbrun et al., (2007) J Virol 81: 2165-2178; Kozak et al., (2006) J Biol Chem 281: 29105-29119). Although in subsequent analysis a direct association between APOBEC3G and Mov10 (data not shown) was not found. Whether perturbation of Mov10 expression could impact HIV-1 infectivity in a manner similar to APOBEC3G was evaluated. Accordingly, 293T cells were transfected with different Mov10 plasmid amounts. Mov10 was highly expressed upon transfection (FIG. 1A), and this expression level was not immediately toxic to cells as no cell death was observed over three days following transfection (data not shown).

Whether viruses produced in the presence of ectopic Mov10 exhibited reduced infectivity similar to that typically observed with APOBEC3G-transfected 293T cells was determined. To address this point, 293T cells were co-transfected with GFP-expressing HIV-1 (with or without Vif), a vesicular somatitis virus glycoprotein (VSV-G) expression construct, and either a Mov10 or APOBEC3G expression plasmid. Two days later, VSV-G pseudotyped HIV-1 vectors produced from the transfected 293T cells were used to infect the Jurkat T cell line. Overexpression of Mov10 almost completely abolished infectivity of HIV-1 produced by 293T cells (FIG. 1B). However, in contrast to APOBEC3G overexpression, the Mov10-mediated reduction of HIV-1 infectivity could not be rescued by co-expression of HIV-1 vif (FIG. 1B). These data demonstrate that increased levels of Mov10 have a profound effect on the generation of infectious HIV-1.

Whether the suppression of HIV-1 infectivity through Mov10 overexpression was due to reduced HIV-1 particle production was investigated. It was found that at varying ratios of plasmids encoding HIV-1 to Mov10, the HIV-1 particle production, as assessed by p24 protein levels in supernatant, was mostly unaffected (FIG. 2A and FIG. 1C); while the infectivity of the viruses normalized to p24 levels were greatly reduced using either luciferase- or GFP-expressing viruses (FIG. 2B, 2C and FIG. 1D). However, at the ratios where Mov10 levels were highest, a notable reduction in the level of p24 generated by producer cells (FIG. 2A and FIG. 1C, lowest HIV-1/Mov10 expressing plasmid ratios) was observed. Similar inhibition of HIV-1 infectivity was observed when viruses were produced from 293T cells stably overexpressing Mov10 for prolonged periods (FIG. 2D).

Whether Mov10 could also inhibit the generation of replication-competent HIV-1 from primary human CD4⁺ T cells was investigated. For this experiment, highly purified CD4⁺ T cells were nucleofected with a replication-competent, CCR5-tropic HIV-1 plasmid (R5.HIV.GFP) in the presence of a Mov10 expression plasmid or control plasmid (pcDNA3). The virus produced from CD4⁺ T cells from this transfection was in turn used to infect CCR5⁺ Hut78 T cells (experimental setup shown in FIG. 3A). Virus production in primary cells was slightly impaired in the presence of Mov10 (FIG. 3B). However, when supernatants containing identical levels of p24 were applied to Hut78 cells, a significant reduction in HIV-1 infectivity due to Mov10 overexpression was observed (FIG. 3C). Next, viral plasmids for other retroviruses, SIV, MLV, FIV and EIAV, along with the Mov10 expression plasmid or empty vector to produce the respective viruses in presence or absence of Mov10 were co-transfected. Their infectivity on HeLa cells was tested and found that in the presence of Mov10, similar to HIV-1, infectivity of all of the tested lentiviruses and the retrovirus were profoundly suppressed (FIG. 4).

Because overexpression of Mov10 impaired HIV-1 infectivity, whether endogenously expressed Mov10 was also inhibitory to HIV-1 replication was investigated. To address this Mov10 expression was silenced in producer cells using siRNAs targeting Mov10. Reduced levels of Mov10 also suppressed HIV-infectivity (FIG. 5), indicating a positive role for Mov10 in the generation of optimally infectious virions. To further test whether Mov10 levels were indeed critical or whether the siRNA was acting “off target,” Mov10 was ectopically expressed in siRNA-treated cells from which HIV-1 was produced. Because high levels of Mov10 would be inhibitory to HIV-1, the knockdown cells were complemented with a wild-type form of Mov10 that remained susceptible to the siRNA pool to restrict increased expression. In the absence of Mov10 siRNA, transfection of increasing amounts of the Mov10 expression plasmid leads to a dramatic increase of Mov10 levels even at relatively low input DNA amounts (FIG. 5A, left). At the highest levels of Mov10 (0.1 μg DNA), the specific infectivity of HIV-1 is diminished (FIG. 5B). In the presence of Mov10 siRNA, Mov10 levels, relative to protein loading controls, are less dramatically increased by Mov10 plasmid transfection (FIG. 5A, right). Without Mov10 plasmid transfection, siRNA targeting of endogenous Mov10 led to a 2-fold reduction in HIV-1 particle infectivity (FIG. 5B). The restoration of Mov10 expression to endogenous levels (0.1 μg DNA) was sufficient to increase HIV-1 specific infectivity. These data indicate that endogenous Mov10 aids in HIV-1 replication and that slight variation from the wild type level of Mov10 can drastically affect the infectivity of HIV-1.

Mov10 decreases infectivity of HIV-1. In order to determine how Mov10 reduces the infectivity of HIV-1, the early events in the lifecycle of HIV-1 produced in the presence of perturbed Mov10 levels were analyzed. Whether Mov10 interfered with the incorporation of viral glycoproteins in HIV-1 particles was determined. Virion-like particles (VLP) that express GFP through fusion to Gag and trans-incorporate VSV-G were constructed. The VLPs were then used to assess their binding capacity to a T cell line by analysis for GFP. When the target cells were analyzed by flow cytometry, there was no decrease in the ability of VLPs produced in the presence of Mov10 to bind to Jurkat cells (FIG. 6). Whether Mov10-treated HIV-1 has defects during its early, post-entry stages was evaluated. Quantitative real-time PCR was used to analyze the early and late HIV-1 reverse transcripts (FIG. 7). Compared to virus from cells expressing a control plasmid, virus from Mov10-overexpressing cells was 80% less efficient in synthesis of early reverse transcripts (minus-strand strong-stop DNA). This indicates that the defect induced by Mov10 manifests prior to the initiation of reverse transcription during the early stages of post-entry replication of the virus.

### EXAMPLE 2

### Materials and Methods

In an effort to study retrotransposon suppression by Mov10, an assay by Esnault et al. (Esnault et al. Nature (2005) 433:430-434) was adapted. Mammalian long-terminal-repeat (LTR) retrotransposons (or endogenous retroviruses) such as murine IAP and MusD sequences or human endogenous retroviruses (HERVs) are structurally similar to infectious retroviruses.

200,000 Hela cells were plated in a 6 well plate a day before transfection at 70-90% confluent monolayer. One well was used for transfection with GFP plasmid alone for control. Transfection mix: 1 μg of retrotransposon plasmid and 0.5 μg of Mov10 plasmid were prepared according to manufacture protocol. Transfection was performed using the lipofectamine 2000 reagent (Invitrogen). 1.5 μg of total diluted DNA was mixed with 4.5 μl of diluted lipofectamine 2000. The complex was added to the cells, and the media was changed the next day. Cells were trypsinized 2 days posttransfection, collected in one tube and then divided in 3 parts. 2 tubes were washed with PBS. 2 tubes were frozen with a cell pellet at −80° C. for DNA and RNA analysis.

The cell pellet from the third tube was used to plate in 2 wells in a 6 well plate (around 150,000 cells). The cells were propagated for another 3 days. 300,000 cells from each well were then plated in 100 mm Petri dishes in 5 ml of DMEM complete media with FBS as per 293t cells. G418 (700 ug/ml) was added for selection until cell foci were visible, usually 10-12 days. The media was changed with fresh G418 every 4 days. The cells were fixed and stained with crystal violet.

IAP, Line-1 and MusD retrotransposon encoding plasmids were described by Esnault et al. Nature (2005) 433:430-434. The plasmids contained a Neo resistance cassette. Mov10 protein was cloned in pCMV6 (Oriegene) or pcDNA3 plasmid (Invitrogen). Mov10 mutants were described by Furtak et al., (2010). PLoS One 5:e9081.

### Results

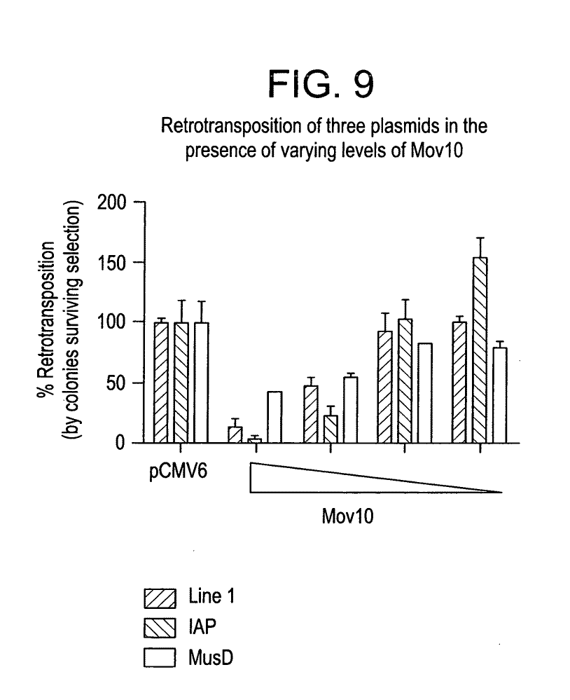

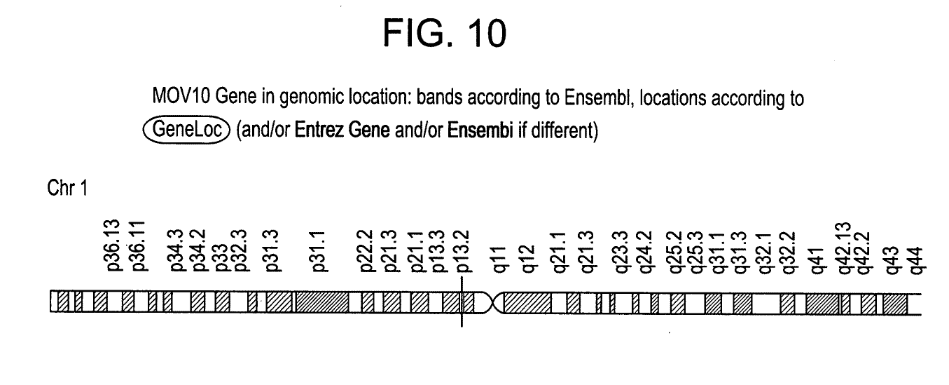

FIG. 9 demonstrates that Mov10 suppresses the activity of LTR and non-LTR retrotransposons. HeLa cells were co-transfected with a retrotransposon construct and Mov10 in ratios of 3:1, 15:1, 30:1, or 750:1 (mass construct:mass Mov10 plasmid) and then reseeded in G418 selection media after 4 days. Only plasmids that were successfully reverse-transcribed and integrated into the genome could allow a cell to survive selection. Cells were subsequently expanded for 14 days under selection and their colonies counted to determine efficiency of retrotransposition. Baseline retrotransposition in cells transfected with only the appropriate retrotransposon construct and a pCMV6 empty vector control is normalized to 100% for each construct. The retrotransposon constructs express the endogenous retroviruses (LTR-containing) IAP (Dewannieux et al., (2004) Nat Genet 36: 534-539), or MusD (Ribet et al., (2004) Genome Res 14: 2261-2267); or the non-LTR Linel construct (Cordaux et al., (2009) Nat Rev Genet 10: 691-703). Error bars represent standard deviation of experiments run in duplicate.

Mov10 cotransfected with IAP, Line-1 and MusD retrotransposon encoding plasmids significantly inhibited efficiency of retrotransposition (FIG. 9). Apobec3A and Mov10 showed a similar degree of inhibition. The number of colonies is proportional to the efficiency of retrovirus incorporation into the cellular genome (retrotransposition). If colonies are not formed, this indicates that expression of Mov10 inhibited retrotransposition.

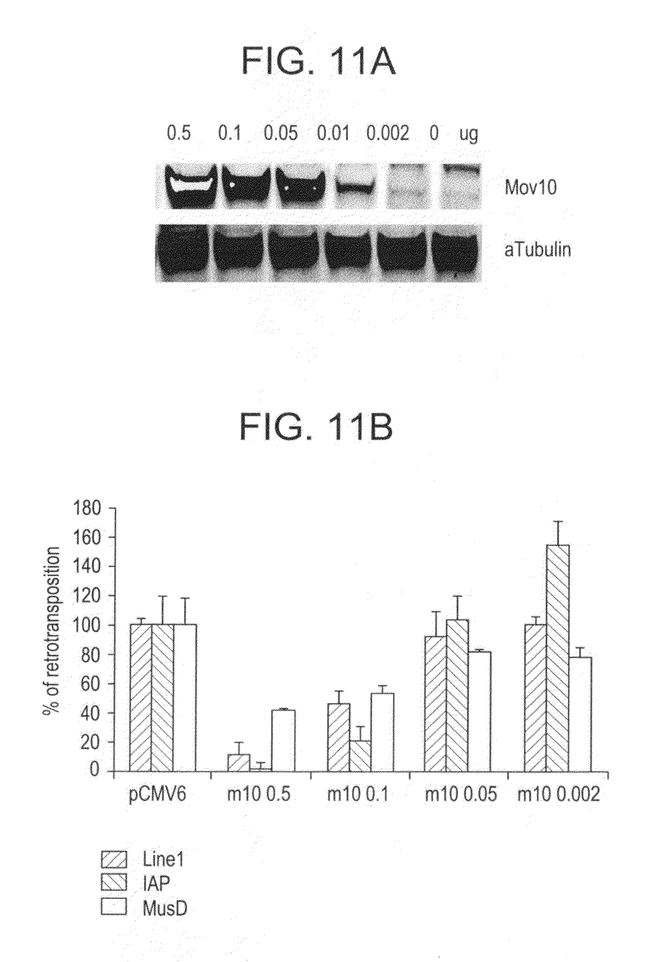

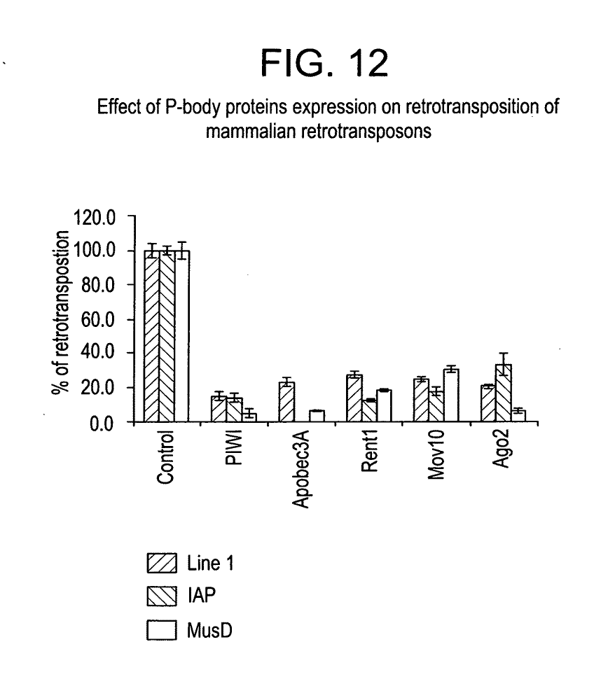

The Mov10 inhibitory effect on retrotransposon integration is dose dependent. HeLa cells were transfected with different amounts of Mov10 expressing plasmids and probed with antibodies against Mov10 in western blot (FIG. 11A). Retrotranspositions were gradually inhibited with an increasing amount of Mov10. IAP retrotransposon showed slightly higher sensitivity to Mov10 in comparison to Line-1 and MusD (FIG. 11B). Other P-body proteins (Ago-2, PIWI and Rent1) also exhibited activity against retrotransposons (FIG. 12).

When expression of Mov10 is silenced or knocked out, HIV infectivity is not only improved but in fact slightly reduced, in some instances about two fold. When Mov10 is overexpressed in 293T cells, HIV infectivity (the output of HIV that is infectious) is significantly suppressed. Thus if Mov10 was a classical restriction factor, higher levels of infectious HIV would occur when Mov10 expression is silenced. Therefore, Mov10 appears not to be a restriction factor for HIV per se. Rather, increasing (or partly decreasing) its levels in cells perturbs the p-body or other complexes in the cells that are required for viral RNA processing. Compounds (small molecules, proteins, peptides, biologicals, antibodies, etc.) that can increase and also decrease Mov10 expression are useful for suppressing HIV infection.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. It is further to be understood that all base sizes or amino acid sizes, and all molecular weight or molecular mass values, given for nucleic acids or polypeptides are approximate, and are provided for description. Various publications are cited herein, the disclosures of which are all incorporated by references in their entireties.

We claim:

1. A method of inhibiting a retrovirus replication or infectivity by administering a therapeutically effective amount of MOV or a fragment, derivative or analog thereof.

2. A method according to claim 1 wherein the retrovirus is HIV.

3. A method according to claim 2 wherein the retrovirus is HIV-1.

4. A method according to claim 1 wherein the fragment, derivative or analog thereof has about 95% sequence homology to SEQ ID NO: 1.

5. A method of inhibiting a retrovirus replication or infectivity by increasing transcription,
translation or biological activity of MOV10 or a fragment, derivative or analog thereof.

6. A method according to claim 5 wherein the retrovirus is HIV.

7. A method according to claim 6 wherein the retrovirus is HIV-1.

8. A method according to claim 5 wherein the fragment, derivative or analog thereof has about 95% sequence homology to SEQ ID NO: 1.

9. A method of treating a disease caused all or in part by a retrovirus comprising administering a therapeutically effective amount of MOV10 or a fragment, derivative or analog thereof or by increasing transcription, translation or biological activity of MOV10 or a fragment, derivative or analog thereof.

10. A method according to claim 9 wherein the retrovirus is HIV.

11. A method according to claim 10 wherein the retrovirus is HIV-1.

12. A method according to claim 9 wherein the fragment, derivative or analog thereof has about 95% sequence homology to SEQ ID NO: 1.

13. A method for identifying an agent that may inhibit a retrovirus comprising identifying an agent that is regulated by MOV10.

14. A method of inhibiting a retrovirus replication or infectivity by administering a therapeutically effective amount of an inhibitor of MOV10 or a fragment, derivative or analog thereof.

15. A method according to claim 14 wherein the retrovirus is HIV.

16. A method according to claim 15 wherein the retrovirus is HIV-1.

17. A method according to claim 14 wherein the fragment, derivative or analog thereof has about 95% sequence homology to SEQ ID NO: 1.

18. A method of inhibiting a retrovirus replication or infectivity by decreasing transcription, translation or biological activity of MOV10 or a fragment, derivative or analog thereof.

19. A method according to claim 18 wherein the retrovirus is HIV.

20. A method according to claim 19 wherein the retrovirus is HIV-1.

21. A method according to claim 18 wherein the fragment, derivative or analog thereof has about 95% sequence homology to SEQ ID NO: 1.

22. A pharmaceutical composition comprising a therapeutically effective amount of MOV10 or a fragment, derivative or analog thereof.

23. A pharmaceutical composition comprising a therapeutically effective amount of an inhibitor of MOV10 or a fragment, derivative or analog thereof.

24. A pharmaceutical composition according to claim 22 wherein the fragment, derivative or analog thereof has about 95% sequence homology to SEQ ID NO: 1.

25. A pharmaceutical composition according to claim 23 wherein the fragment, derivative or analog thereof has about 95% sequence homology to SEQ ID NO: 1.

26. A method of inhibiting efficiency of retrovirus incorporation into a cellular genome or retrotransposition by administering a p-body protein or a biologically active fragment, derivative or analog thereof or by increasing transcription, translation or biological activity of a p-body protein or a biologically active fragment, derivative or analog thereof.


SEQUENCE LISTING (9 sequences)

SEQ ID NO: 1 - protein, 1003 aa, Moloney leukemia virus
MPSKFSCRQLREAGQCFESFLVVRGLDMETDRERLRTIYNRDFKISFGTPAPGFSSMLYGMKIANLAYVTKTRVRFFRLDRWADVRFPEKRRMKLGSDISKHHKSLLAKIFYDRAEYLHGKHGVDVEVQGPHEARDGQLLIRLDLNRKEVLTLRLRNGGTQSVTLTHLFPLCRTPQFAFYNEDQELPCPLGPGECYELHVHCKTSFVGYFPATVLWELLGPGESGSEGAGTFYIARFLAAVAHSPLAAQLKPMTPFKRTRITGNPVVTNRIEEGERPDRAKGYDLELSMALGTYYPPPRLRQLLPMLLQGTSIFTAPKEIAEIKAQLETALKWRNYEVKLRLLLHLEELQMEHDIRHYDLESVPMTWDPVDQNPRLLTLEVPGVTESRPSVLRGDHLFALLSSETHQEDPITYKGFVHKVELDRVKLSFSMSLLSRFVDGLTFKVNFTFNRQPLRVQHRALELTGRWLLWPMLFPVAPRDVPLLPSDVKLKLYDRSLESNPEQLQAMRHIVTGTTRPAPYIIFGPPGTGKTVTLVEAIKQVVKHLPKAHILACAPSNSGADLLCQRLRVHLPSSIYRLLAPSRDIRMVPEDIKPCCNWDAKKGEYVFPAKKKLQEYRVLITTLITAGRLVSAQFPIDHFTHIFIDEAGHCMEPESLVAIAGLMEVKETGDPGGQLVLAGDPRQLGPVLRSPLTQKHGLGYSLLERLLTYNSLYKKGPDGYDPQFITKLLRNYRSHPTILDIPNQLYYEGELQACADVVDRERFCRWAGLPRQGFPIIFHGVMGKDEREGNSPSFFNPEEAATVTSYLKLLLAPSSKKGKARLSPRSVGVISPYRKQVEKIRYCITKLDRELRGLDDIKDLKVGSVEEFQGQERSVILISTVRSSQSFVQLDLDFNLGFLKNPKRFNVAVTRAKALLIIVGNPLLLGHDPDWKVFLEFCKENGGYTGCPFPAKLDLQQGQNLLQGLSKLSPSTSGPHSHDYLPQEREGEGGLSLQVEPEWRNEL

SEQ ID NO: 2 - DNA, 36 bp, Artificial Sequence
  Description of Artificial Sequence Synthetic primer
tacgccggatcccccagtaagttcagctgccggcag

SEQ ID NO: 3 - DNA, 42 bp, Artificial Sequence
  Description of Artificial Sequence Synthetic primer
cgttaggcggccgctcagagctcattcctccactctggctcc

SEQ ID NO: 4 - DNA, 36 bp, Artificial Sequence; identical to SEQ ID NO: 2
  Description of Artificial Sequence Synthetic primer

SEQ ID NO: 5 - DNA, 42 bp, Artificial Sequence
  Description of Artificial Sequence Synthetic primer
cgttaggcggccgctcaccggtcgtacagcttgagtttcaca

SEQ ID NO: 6 - DNA, 36 bp, Artificial Sequence
  Description of Artificial Sequence Synthetic primer
tacgccggatccagtctggagtcaaacccagagcag

SEQ ID NO: 7 - DNA, 42 bp, Artificial Sequence; identical to SEQ ID NO: 3
  Description of Artificial Sequence Synthetic primer

SEQ ID NO: 8 - protein, 4 aa, Moloney leukemia virus
DEAG

SEQ ID NO: 9 - protein, 4 aa, Moloney leukemia virus
DQAG